(19) United States
(12) Patent Application Publication
(10) Pub. No.: US 2021/0333263 A1
(43) Pub. Date: Oct. 28, 2021

(54) REDOX ACTIVATABLE FLUORESCENT SENSORS

(71) Applicant: The Board of Regents of the University of Texas System, Austin, TX (US)

(72) Inventors: Jinming GAO, Plano, TX (US); Zhaohui WANG, Dallas, TX (US); Min LUO, Irving, TX (US); Qi WEI, Dallas, TX (US); Gang HUANG, Plano, TX (US)

(21) Appl. No.: 16/333,164

(22) PCT Filed: Sep. 12, 2017
(86) PCT No.: PCT/US2017/051075

§ 371 Date: Mar. 13, 2019

Related U.S. Application Data
(60) Provisional application No. 62/395,834, filed on Sep. 16, 2016.

(51) Int. Cl. G01N 33/50; G01N 33/542; C09K 11/07; G01N 21/64

(52) U.S. Cl. CPC G01N 33/502; G01N 33/542; C09K 11/07; G01N 2021/6439; G01N 2500/10; G01N 2021/6432; G01N 21/6428

(57) ABSTRACT

Provided herein are compounds of the formula: (I) wherein the variables are defined herein, which may be used as redox-activatable sensors. In some aspects, the compounds are fluorescence sensors which are activated by the redox state of the cell. Also provided herein are methods of using these compounds to image the cellular environment or the delivery of macromolecules to a cell.

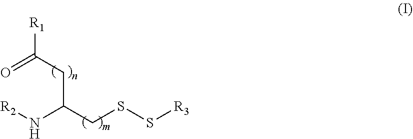

This application claims benefit of priority to U.S. Provisional Application Ser. No. 62/395,834, filed Sep. 16, 2016, the entire contents of which are hereby incorporated by reference.

This invention was made with government support under Grant No. R01 EB013149 awarded by the National Institutes of Health. The government has certain rights in the invention.

### BACKGROUND

### 1. Field

The present disclosure relates generally to the field of fluorescence imaging. More particularly, it concerns fluorescent imaging using redox-activatable fluorescent molecules. These fluorescent imaging molecules may be used to monitor the cytosolic delivery of macromolecules.

### 2. Description of Related Art

Cytosolic delivery of biomacromolecules (e.g., proteins, peptides, nucleic acids) is critically important to achieve biological efficacy in immunotherapy, gene therapy and RNA interference (Kong and Mooney 2007, Lachelt and Wagner 2015 and Castanotto 2009). Macromolecular agents are typically taken up by the target cells through endocytosis or macropinocytosis, where escape from endolysosomes is essential to prevent proteolytic degradation inside the lysosomes (Bareford 2007 and Whitehead et al., 2009). To achieve this goal, extensive efforts have been devoted to the development of cytosolic delivery strategies that allow endolysosomal escape of biomacromolecules to reach their targets in the cytoplasm (Pack et al., 2005, Gu et al., 2011 and Jiang et al., 2015)

Despite great advances, the lack of a broad and quantitative detection assay presents a major challenge in the rapid discovery of new strategies for the cytosolic delivery of macromolecules with different size, charge and physical properties (Holub et al., 2013 and Deng et al., 2015). Conventional method utilizes confocal microscopy to investigate the spatial and temporal distribution of fluorescently labeled macromolecules (Gilleron et al. and Sahay et al., 2013). Typical fluorescent labels employ “always on” reporter molecules where detection intensity is solely dependent on the probe concentration. Such imaging strategies have low signal-to-noise ratio and lack detection accuracy due to the extensive dilution of probe in the cytosol, strong signal in the endocytic vesicles, and a confounding effect of cytosolic autofluorescence background. Furthermore, they are not compatible with high-throughput assays such as plate readers when quantification of subcellular distribution is not feasible. Therefore, a simple, quantifiable cytosolic sensing assay is urgently needed to achieve high-throughput screening and microscopic examination of endolysosomal escape and cytosolic delivery of macromolecules in living cells. Therefore, there remains a need for new imaging agents particular those which are responsive to the redox state of the environment.

### SUMMARY

In some aspects, the present disclosure provides compounds of the formula:

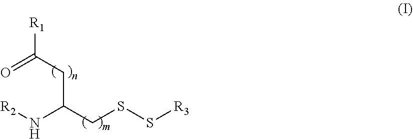

wherein:
R₁ is a first label, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, an amine reactive group; a thiol reactive group, an organic polymer, a nucleic acid sequence, a peptide, or a protein;
R₂ is a first label, hydrogen, alkyl_{(C≤8)}, substituted alkyl_{(C≤8)}, an organic polymer, a nucleic acid sequence, a peptide, or a protein;
R₃ is a second label; and
m and n are each independently 0, 1, 2, or 3;
provided that either R₁ or R₂ is a first label.
   In some embodiments, the compounds are further defined as:

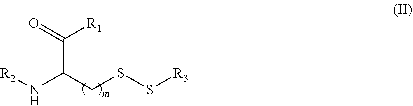

wherein m, R₁, R₂, and R₃ are as defined above.
   In some embodiments, the compounds are further defined as:

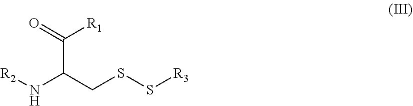

wherein R₁, R₂, and R₃ are as defined above.

In some embodiments, R₂ or R₃ is a fluorescent dye. R₂ and R₃ may both be fluorescent dyes. In some embodiments, R₂ and R₃ are different fluorescent dyes. R₂ and R₃ may both be different fluorescent dyes which form a Forster resonance energy transfer (FRET) pair. In some embodiments, R₂ is the FRET pair donor or acceptor. In some embodiments, R₃ is the FRET pair acceptor or donor. R₂ or R₃ may be a xanthene dye such as a rhodamine dye. In other embodiments, R₂ or R₃ is a cyanine dye. R₂ or R₃ may be a coumarin dye. In other embodiments, R₂ or R₃ is a BODIPY dye. In other embodiments, R₂ or R₃ is a fluorescent quencher such as BHQ-1, BHQ-2, BHQ-3, QSY21, QSY35, or DABCYL. In some embodiments, R₂ or R₃ is a fluorescent dye and the other is a fluorescent quencher.

In some embodiments, m is 1 or 2. In some embodiments, n is 0 or 1. R₁ may be hydroxy and thereby forms a carboxylic acid. In other embodiments, R₁ is an amine reactive group such as N-hydroxysuccinimide.

R₁ may be an organic polymer, a nucleic acid sequence, a peptide, or a protein. In some embodiments, R₁ is a protein such as ovalbumin, lysozyme, histone, or immunoglobulin G. In other embodiments, R₁ is an organic polymer such as an organic polymer of the formula:

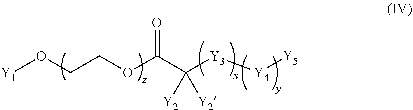

wherein:
Y₁ is hydrogen, alkyl_{(C≤12)}, cycloalkyl_{(C≤12)}, substituted alkyl_{(C≤12)}, substituted cycloalkyl_{(C≤12)}, a cell targeting moiety, or a conjugating group;
z is an integer from 1 to 500;
Y₂ and Y₂′ are each independently selected from hydrogen, alkyl_{(C≤12)}, cycloalkyl_{(C≤12)}, substituted alkyl_{(C≤12)}, or substituted cycloalkyl_{(C≤12)};
Y₃ is a group of the formula:

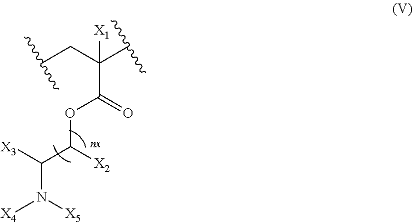

wherein:
nₓ is 0-3;
X₁, X₂, and X₃ are each independently selected from hydrogen, alkyl_{(C≤12)}, cycloalkyl_{(C≤12)}, substituted alkyl_{(C≤12)}, or substituted cycloalkyl_{(C≤12)}; and
X₄ and X₅ are each independently selected from alkyl_{(C≤12)}, cycloalkyl_{(C≤12)}, or a substituted version of any of these groups, or X₄ and X₅ are taken together and are alkanediyl_{(C≤12)}, alkoxydiyl_{(C≤12)}, alkylaminodiyl_{(C≤12)}, or a substituted version of any of these groups;
x is an integer from 1 to 150;
Y₄ is a group of the formula:

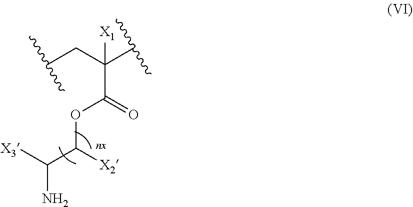

wherein:
n_{y} is 0-3; and
X₁′, X₂′, and X₃′ are each independently selected from hydrogen, alkyl_{(C≤12)}, cycloalkyl_{(C≤12)}, substituted alkyl_{(C≤12)}, or substituted cycloalkyl_{(C≤12)};
y is an integer from 1 to 20; and
Y₅ is hydrogen, halo, hydroxy, alkyl_{(C≤12)}, or substituted alkyl_{(C≤12)}.

In some embodiments, the compound is further defined as:

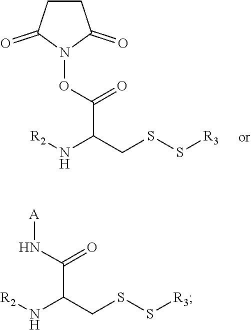

wherein:
R₂ and R₃ are each independently a fluorescent dye or a fluorescent quencher; and
A is a peptide, protein, or nucleic acid.

The protein may be immunoglobin G or ovalbumin. In some embodiments, the fluorescent dye is tetramethylrhodamine, cyanine 5, 7-diethylaminocoumarin, 7-hydroxycoumarin, or BODIPY-493. The fluorescent quencher may be QSY355, DABCYL, BHQ-1, BHQ-2, or BHQ-3.

In another aspect, the present disclosure provides compositions comprising:
(a) a compound described herein; and
(b) a polymer;
   wherein the polymer forms a micelle. In some embodiments, the polymer may be a compound of the formula:

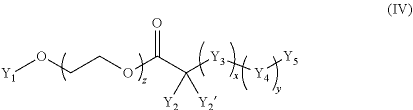

wherein:
Y₁ is hydrogen, alkyl_{(C≤12)}, cycloalkyl_{(C≤12)}, substituted alkyl_{(C≤12)}, substituted cycloalkyl_{(C≤12)}, a cell targeting moiety, or a conjugating group;
n is an integer from 1 to 500;
Y₂ and Y₂′ are each independently selected from hydrogen, alkyl_{(C≤12)}, cycloalkyl_{(C≤12)}, substituted alkyl_{(C≤12)}, or substituted cycloalkyl_{(C≤12)};
Y₃ is a group of the formula:

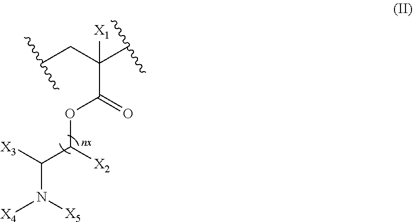

wherein:
nₓ is 0-3;
X₁, X₂, and X₃ are each independently selected from hydrogen, alkyl_{(C≤12)}, cycloalkyl_{(C≤12)}, substituted alkyl_{(C≤12)}, or substituted cycloalkyl_{(C≤12)}; and
X₄ and X₅ are each independently selected from alkyl_{(C≤12)}, cycloalkyl_{(C≤12)}, or a substituted version of any of these groups, or X₄ and X₅ are taken together and are alkanediyl_{(C≤12)}, alkoxydiyl_{(C≤12)}, alkylaminodiyl_{(C≤12)}, or a substituted version of any of these groups;
x is an integer from 1 to 150;
Y₄ is a group of the formula:

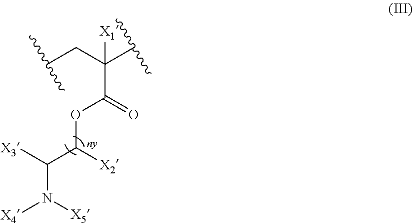

wherein:
n_{y} is 0-3;
X₁′, X₂′, and X₃′ are each independently selected from hydrogen, alkyl_{(C≤12)}, cycloalkyl_{(C≤12)}, substituted alkyl_{(C≤12)}, or substituted cycloalkyl_{(C≤12)}; and
X₄′ and X₅′ are each independently selected from hydrogen, alkyl_{(C≤12)}, cycloalkyl_{(C≤12)}, acyl_{(C≤12)}, substituted alkyl_{(C≤12)}, substituted cycloalkyl_{(C≤12)}, substituted acyl_{(C≤12)}, a dye, or a quencher;
y is an integer from 0 to 20; and
Y₅ is hydrogen, halo, hydroxy, alkyl_{(C≤12)}, or substituted alkyl_{(C≤12)}.

In still yet another aspect, the present disclosure provides methods of determining the redox state of a composition comprising:
(a) contacting the composition with a compound or pharmaceutical composition described herein; and
(b) measuring for a signal from the each of the labels of the compound which indicates a reducing environment.

The composition may be a cell. In other embodiments, the composition is a human body. The composition may be a composition outside of a living organism such as one carried out in a test tube experiment, on a bench top, or in other in vitro methods. Additionally, one of the label may allow the imaging of the location of the compound in the composition.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows high-throughput assay of cytosolic delivery of biomacromolecules labeled with a redox-activatable sensor using a plate reader. The fluorescence signal is off in the extracellular environment and through the endocytic pathway. After endolysosomal disruption and reaching to the cell cytosol, the fluorescence signal is turned on by cytosolic GSH activation.

FIGS. 2A-2C show the synthesis and characterization of a representative qRAS molecule. (FIG. 2A) Scheme of qRAS synthesis using TMR and Cy5 as model fluorescent donor and acceptor, respectively. (FIG. 2B) The fluorescence spectra of qRAS after the addition of TCEP to cleave the disulfide bond. The sample was excited at 550 nm. (FIG. 2C) Ratio of F₅₇₅/F₆₆₅ over time in response to redox cleavage by TCEP (diamonds). Data was obtained from (FIG. 2B). The emission intensity of Cy5 (F₆₆₅, excited at 640 nm; squares) was used for comparison. Both F₅₇₅/F₆₆₅ and F₆₆₅ values were normalized to those at t=0.

FIG. 3 shows the UV-Vis absorbance spectrum of qRAS. qRAS was dissolved in methanol, the absorbance was recorded on UV-Vis spectrometer from 450 to 750 nm. The peaks at 550 nm and 645 nm represent the absorbance of donor dye (TMR) and acceptor dye (Cy5), respectively.

FIGS. 4A & 4B show the fluorescence emission spectra of two qRAS probes with switched donor/acceptor positions before and after the addition of TCEP to cleave the disulfide bond in methanol. The samples were excited at 550 nm.

FIGS. 5A-5C show the emission spectra of qRAS probes 7-diethylaminocoumarin/QSY35 (FIG. 5A), 7-hydroxycoumarin/Dabcyl (FIG. 5B) and BODIPY 493/BHQ-1 quencher (FIG. 5C) before and after the addition of reducing reagent TCEP on a Hitachi fluorometer.

FIG. 6 show the emission spectra of qRAS probe (7-hydroxycoumarin/Dabcyl) as a function of pH in the PBS buffer. Samples were excited at 390 nm.

FIGS. 7A-7F show (FIG. 7A) Schematic of qRAS conjugation to OVA and redox activation by GSH in the cytosol. (FIG. 7B) Emission spectra of qRAS-labeled OVA (OVA^{qRAS}) before and after the addition of GSH (5 mM) in PBS. Samples were excited at 550 nm. (FIG. 7C) Fluorescent images of OVA^{qRAS} solution with and without GSH by a Maestro Imaging system. (FIG. 7D) Normalized fluorescence activation of OVA^{qRAS} over time in response to a redox stimulus. (FIG. 7E) Real-time monitoring of cytosolic delivery efficiency of OVA^{qRAS} by JetPEI and PEG-PLA in the A549 lung cancer cells (n=3). (FIG. 7F) A549 cells were pretreated with NEM before incubation with JetPEI and OVA^{qRAS}. The activation percentage is normalized to the control cells without NEM treatment (n=3; **: p<0.01).

FIG. 8 show the fluorescence emission spectra of OVA^{qRAS} before and after the addition of 1 mM dithiothreitol (DTT) to trigger the cleavage of the disulfide bond. The excitation wavelength was at 550 nm.

FIG. 9 show the normalized fluorescence emission ratio of OVA^{qRAS} in PBS at different pH. OVA^{qRAS} (0.5 mg/mL) was incubated in PBS with different pH for 6 hours. The fluorescence emission spectra of TMR and Cy5 were recorded using a Hitachi fluorometer with excitation wavelength at 550 nm and 640 nm, respectively.

FIG. 10 shows the investigation of redox activation of OVA^{qRAS} by GSH over time on a Tecan plate reader (Infinite 200 PRO). OVA^{qRAS} (0.5 mg/mL) was incubated in PBS solution (pH 7.4) containing 5 mM GSH. Fluorescence emission of TMR was normalized to maximum intensity and plotted versus time.

FIG. 11 shows the linear correlation of fluorescence intensity of OVA^{qRAS} as a function of probe concentration on a plate reader. Fluorescence intensity was measured in the TMR channel as excited at 545 nm. OVA^{qRAS} solution before and after the addition of DTT was shown. The linear correlations of the off and on states of OVA^{qRAS} were used to quantify the activation percentage in live cell imaging applications.

FIG. 12 shows that JetPEI enhanced the cytotoxicity of ribonuclease A. A549 cells were exposed to JetPEI and PEG-PLA (100 μg/mL) with ribonuclease A-aco (5 μg/mL) for 40 min at 37° C. The cell viability was evaluated by the MTT assay after 48 hours incubation. Error bars represent standard deviation of 3 replicate samples.

FIGS. 13A & 13B show the high-throughput screening of cytosolic delivery of OVA^{qRAS} by UPS polymeric nanoparticles on a plate reader. (FIG. 13A) Chemical structures of UPS copolymers PEG-b-P(R₁-r-R₂) with finely tunable hydrophobicity and pKₐ. (FIG. 13B) Cytosolic delivery efficiency of the polymers at 24 hr after co-incubation with OVA^{qRAS} in A549 cells for 40 min (n=3; **: p<0.01).

FIG. 14 shows the synthesis of qRAS-labeled UPS copolymers. The PR segment consists of a random block from two monomers with different molar fractions to fine-tune its pH transition (see Table 1). (R₁ or R₂=Et, ethyl; Pr, propyl; Bu, butyl; Pe, pentyl).

FIG. 15 shows the evaluation of the cytotoxicity of polymers in A549 lung cancer cells. A549 cells were exposed to different polymers (100 μg/mL) for 40 min at 37° C. The cell viability was evaluated by the MTT assay after 48 hours incubation. Error bars represent standard deviation from 3 replicate samples.

FIGS. 16A-16C show the high-throughput quantification of cytosolic delivery efficiency using qRAS-labeled IgG. (FIG. 16A) Scheme of qRAS conjugation to IgG. (FIG. 16B) The fluorescence emission spectra of IgG^{qRAS} before and after the addition of 5 mM GSH. The excite wavelength is 550 nm. (FIG. 16C) The cytosolic delivery efficiency of the polymers at 24 hr after co-incubation with IgG^{qRAS} for 40 min.

FIGS. 17A & 17B show the confocal microscopy analysis of endolysosomal escape and cytosolic delivery of IgG in live cells. A549 cancer cells were co-incubated with IgG^{qRAS} and UPS_{4.4} (FIG. 17A) or PC7A (FIG. 17B) for 40 min. Both the donor TMR (Green) and acceptor Cy5 (Red) signals were recorded over time. Scale bars=10 μm.

FIGS. 18A & 18B show the subcellular imaging of cytosolic activation of OVA^{qRAS} by confocal microscopy. A549 cancer cells were co-incubated with OVA^{qRAS} (25 μg/mL) and UPS_{4.4} (FIG. 18A) or PC7A (FIG. 18B) at 100 μg/mL for 40 min. Both the intracellular fluorescence images of TMR (Green) and Cy5 (Red) channel were taken at different times. Scale bars=10 μm.

FIG. 19A-19E show the broad utility of qRAS in conjugation to various biomacromolecules. Lysozyme (FIG. 19A), histone (FIG. 19B), PC7A (FIG. 19C), UPS_{4.4} (FIG. 19D) and polyethylenimine (FIG. 19E, Branched, 25K Da, BPEI) were used for qRAS conjugation, and demonstrated robust redox activation in response to a reducing reagent. Samples were excited at 550 nm.

### DESCRIPTION OF ILLUSTRATIVE EMBODIMENTS

The present disclosure provides quantitative redox-activatable sensors which may be linked to one or more macromolecules. In some embodiments, these sensors may contain a FRET pair which allows both constant fluorescent signal for monitoring the location and diffusion of the sensor as well as a second fluorescent signal which is activated in the presence of a reducing environment such as the cytosol of a cell. These sensors may be used to image cellular environments to determine the presence of a reducing environment.

### I. Sensors of the Present Disclosure

The sensors provided by the present disclosure are shown, for example, above in the Summary and in the claims below. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Sensors of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. The sensors may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent sensors of the disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the sensors of the present disclosure are intended to include all isotopic forms of such atoms. Isotopes, as used herein, include those atoms having the same atomic number but different mass numbers. By way of general example and without limitation, isotopes of hydrogen include tritium and deuterium and isotopes of carbon include ¹³C and ¹⁴C.

It should be recognized that the particular anion or cation forming a part of any salt form of a sensors provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the sensors provided herein, including any solvates thereof are within the scope of the present disclosure.

### II. Redox State of Biological Systems

The redox state of different biological systems are useful diagnostic signals of the in vivo development of the system. In particular, the consumption of sugar or the production of sugar from light are fundamentally driven by the change in the oxidation state of NADH to NAD⁺. Similarly, the proton gradient within a cell is used to power the production of ATP from AMP and ADP. When reviewing the redox state of a biological system, the redox state is often determined as a balance of molecules such as GSH vs. GSSG, NAD⁺ vs. NADH, or NADP⁺ vs. NADPH, but can be determined based upon other metabolites such as lactate, pyruvate, β-hydroxybutyrate, or acetoacetate.

In general, the extracellular environment is often traditionally in an oxidizing environment while the cytosol is typically a reducing environment. Previous studies have reported that cell cytosol is a reducing environment with high concentrations (1-10 mM) of glutathione (GSH) and neutral pH (7.4) that are optimal to reduce disulfides or reactive oxygen species (Rietsch et al., 1998 and Schafer and Buettner, 2001). In contrast, the endocytic vesicles are much more oxidative with 100-fold lower concentration of GSH and acidic pH as low as 4.5. As a result, disulfide bonds from exogenic molecules are stable in the endosomes or lysosomes, but can be efficiently cleaved in the cell cytosol (Feener et al., 1990 and Go, and Jones, 2008). Therefore, compounds that are sensitive to the redox state of the cell under a chemical transformation which can function to generate a signal of the presence of reducing environment. In some embodiments, the present disclosure provides compounds which under a chemical transformation to signal a reducing environment.

### II. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, for example, the formula

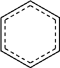

includes

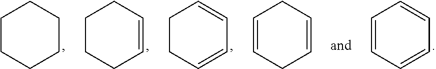

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g. [symbol] for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

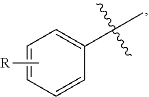

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

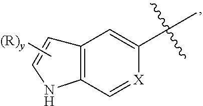

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

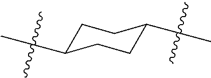 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

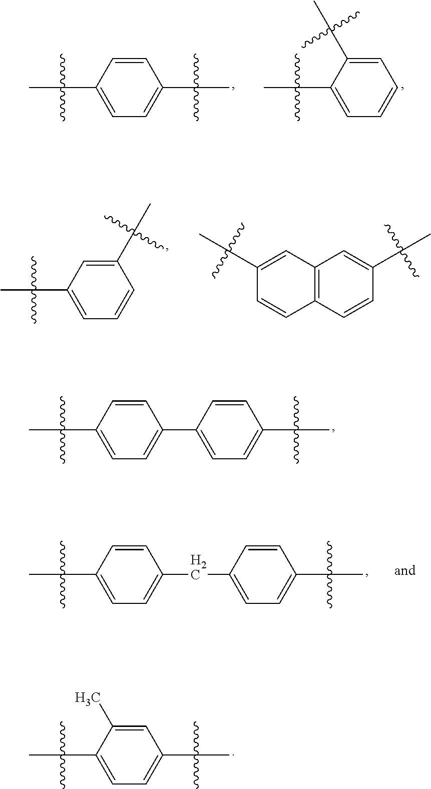

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, or aryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), and —OC(CH₃)₃ (tert-butoxy). The terms “cycloalkoxy”, “alkenyloxy”, “cycloalkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, cycloalkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The terms “alkylthio”, “cycloalkylthio”, and “acylthio” when used without the “substituted” modifier refers to the group —SR, in which R is an alkyl, cycloalkyl, and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy or cycloalkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can each independently be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “cycloalkylamino”, “alkenylamino”, “cycloalkenylamino”, “alkynylamino”, “arylamino”, “aralkylamino”, “heteroarylamino”, “heterocycloalkylamino” and “alkylsulfonylamino” when used without the “substituted” modifier, refers to groups, defined as —NHR, in which R is alkoxy, cycloalkyl, alkenyl, cycloalkenyl, alkynyl, aryl, aralkyl, heteroaryl, heterocycloalkyl, and alkylsulfonyl, respectively. A non-limiting example of an arylamino group is —NHC6H5. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The term “amine reactive group” refers to a chemical functional group which undergoes a reaction with the nitrogen atom of an amino, a primary amine, or a secondary amine to form a covalent bond to the nitrogen atom. In some embodiments, this functional group may be undergo this reaction under any condition which does not result in the degradation of the other functional groups in the molecule. These conditions may include reactions which occur at room temperature and during a time period from a few minutes to a few days. Some non-limiting examples of amine reactive groups include a halo functional group such as a Br atom, a Cl atom, or an I atom, an activated carboxylic acid group, such as an N-hydroxysuccinimidic ester, or a leaving group such as mesylate, tosylate, or triflate.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “cell targeting moiety” is a chemical group which increases the affinity of the compound or composition for a cell. Some non-limiting examples include a molecule which is recognized by a receptor on the surface of the cell (e.g., folate or EGFR) or may be a protein, antibody, or a nucleic acid sequence such as an aptamer.

The term “conjugating group” refers to a chemical functional group which undergoes a reaction with a reactive group of a second molecule such as an amine, carboxylic acid, or a thiol to form a bond. One non-limiting example is a maleimide which undergoes a reaction with a thiol to form a covalent bond.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose that is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The term “protein” refers to an antibody or a protein. The protein may be a wild-type protein or a recombinant protein.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### IV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Development of a Quantitative Redox-Activatable Sensor (qRAS)

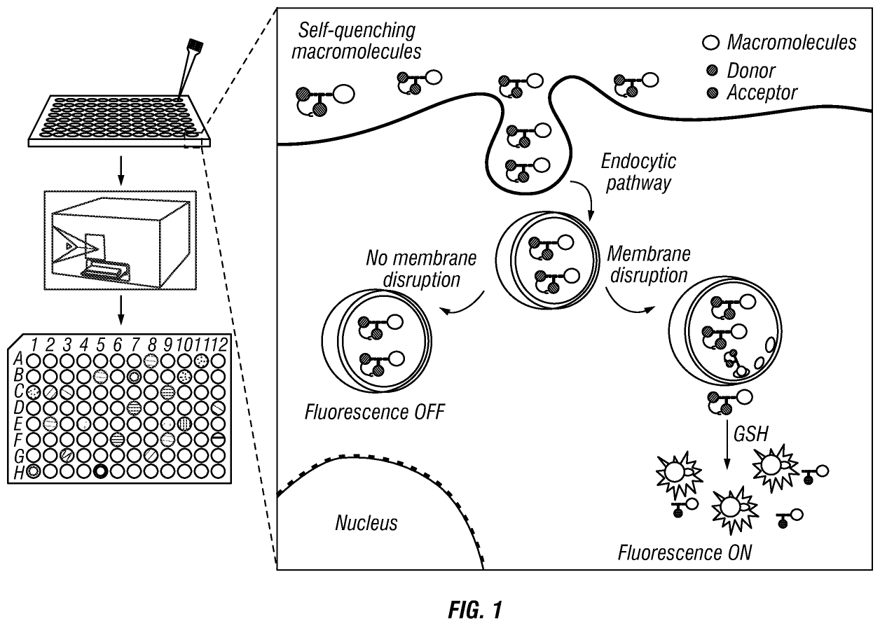

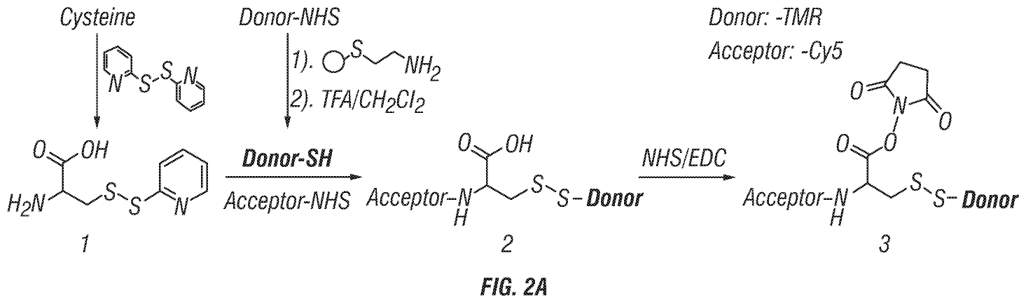

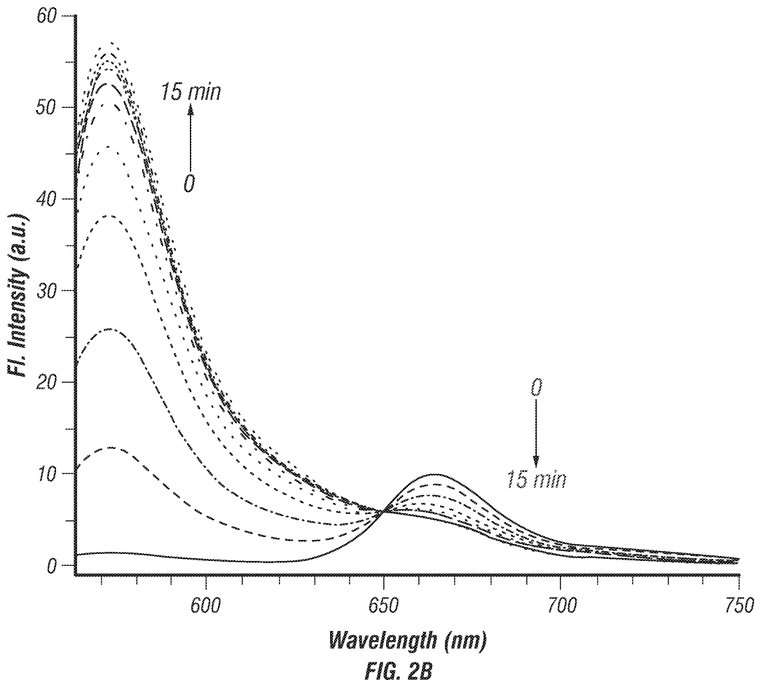

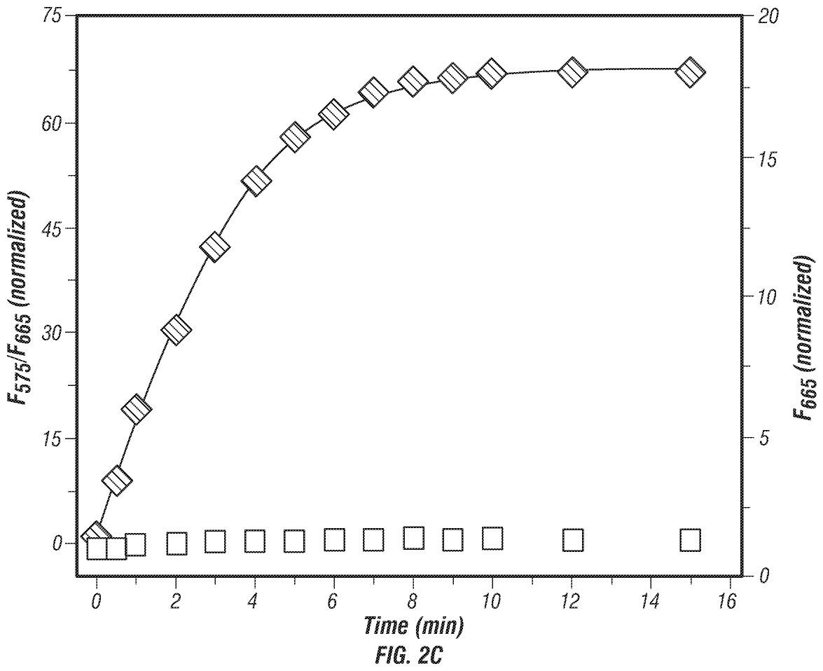

A disulfide-based, redox-activatable fluorescent sensor was developed such that the sensor stays off in the extracellular space and along the endocytic pathway, but can be turned on after endolysosomal disruption and reaching the cell cytosol (FIG. 1). The quantitative redox-activatable sensor (qRAS) is synthesized by conjugating a fluorescent donor and acceptor pair onto the same cysteine (Cys) residue, one of which is through a disulfide bond (FIG. 2A). At the off state with an intact disulfide bond, hetero-Forster resonance energy transfer (hetero-FRET) abolishes the fluorescence signals of the donor dye. Upon disulfide cleavage by GSH in the cell cytosol, the fluorescence signal is turned on as a result of donor/acceptor separation. The donor-Cys-acceptor design maximizes atom efficiency while allowing short distance of donor and acceptor molecules to achieve superior FRET quenching efficiency (Roy et al., 2008). Moreover, the available carboxylic acid group on the Cys residue can be activated by the formation of N-hydroxysuccinimide (NHS) esters and offers a convenient strategy to conjugate qRAS to various amine-containing macromolecules.

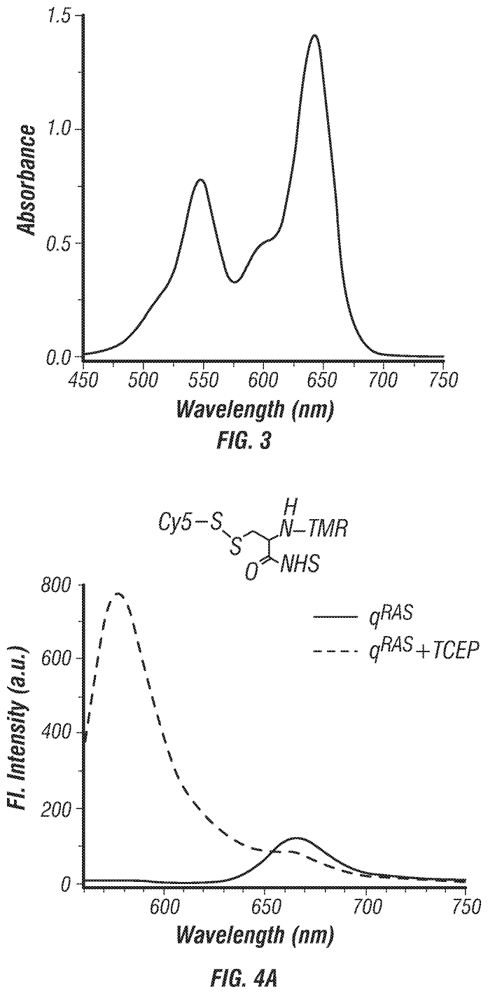

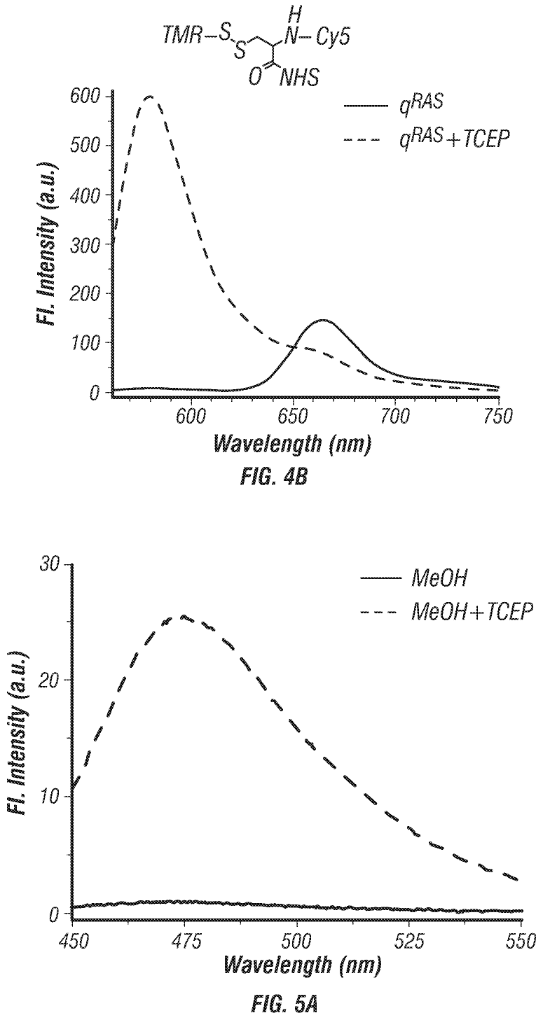

FIG. 2A illustrates the synthesis of an exemplary qRAS molecule, where tetramethyl rhodamine (TMR) and Cyanine 5 (Cy5) were used as donor and acceptor, respectively. After preparation and characterization of the qRAS (FIG. 3), its fluorescence activation in response to a disulfide-reducing agent, tris(2-carboxyethyl)phosphine (TCEP), was investigated. Before TCEP addition, fluorescence spectrum showed nearly extinguished emission at 575 nm for the donor TMR dye (FIG. 2B). The FRET quenching efficiency was calculated to be >97%. Upon addition of 5 mM TCEP, a dramatic increase of the emission intensity at 575 nm was observed, which was accompanied by a decrease in the emission intensity at 665 nm. These results are consistent with the cleavage of the disulfide bond of qRAS, which abolishes the FRET from TMR to Cy5. Plot of F₅₇₅/F₆₆₅ as a function of time showed a short half-activation time (t_{1/2}) at 3 mins and an impressive 70-fold activation ratio over the initial state (FIG. 2C). In contrast, cleavage of TMR has much less pronounced effect (<1.4 fold) on the emission intensity of acceptor Cy5 (F₆₆₅) when irradiated at 640 nm (FIG. 2C). The relatively redox-insensitive acceptor signal is valuable to track labeled macromolecules during cell uptake studies. Similar FRET quenching and redox activation ratios were obtained when the donor and acceptor positions were switched (FIG. 4).

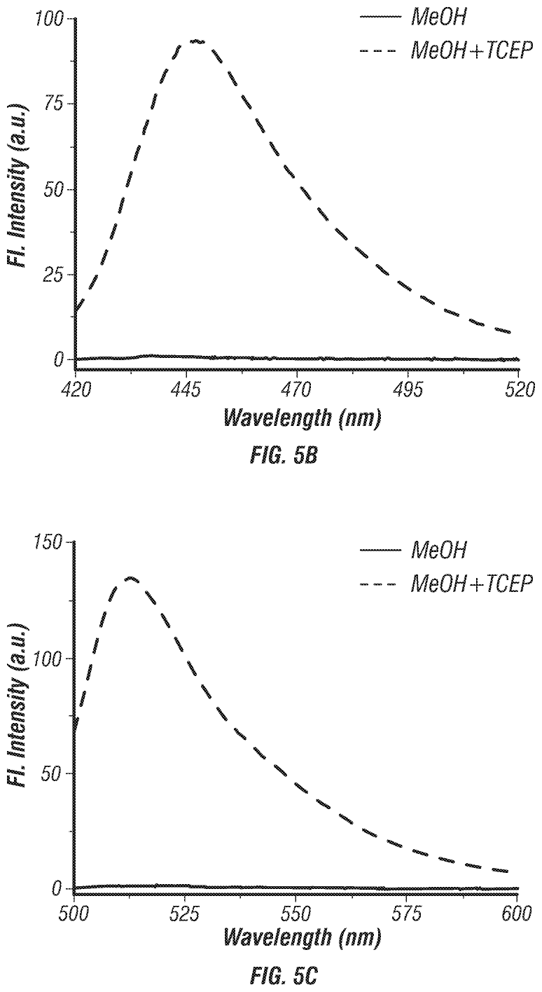

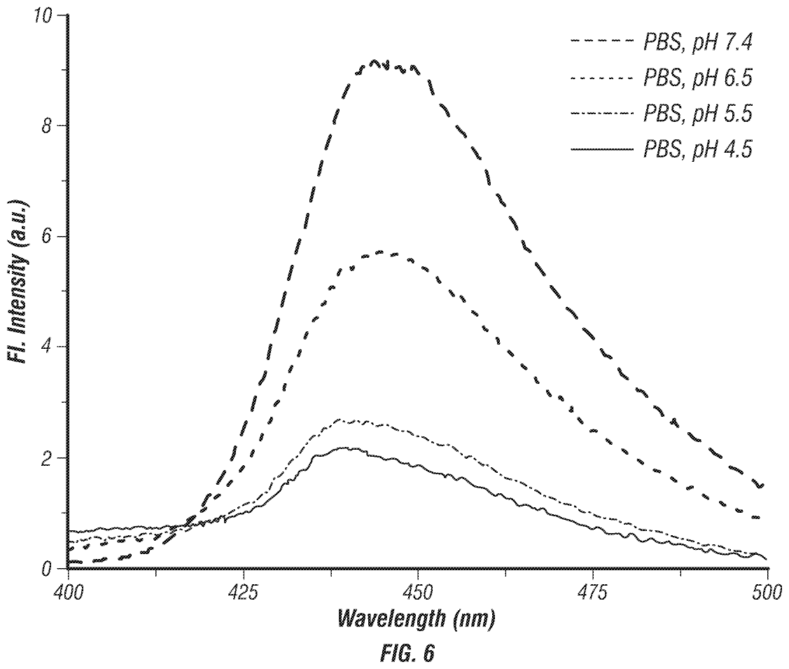

Besides TMR/Cy5, qRAS design was also extended to additional donor/acceptor pairs, including 7-diethylaminocoumarin/QSY35, 7-hydroxycoumarin/dabcyl, and BODIPY-493/BHQ-1 quencher (Johansson, 2006). All exhibited efficient fluorescence activation in response to a reducing agent (FIG. 5). In addition, 7-hydroxycoumarin/dabcyl pair also took advantage of the pH sensitivity of the donor 7-hydroxycoumarin, where the fluorescence signal is further suppressed in the acidic environment such as endocytic organelles (FIG. 6) (Lee et al., 2010). The dual sensitivity design has the potential to further enhance the fluorescence signal in cell cytosol over endocytic organelles. The demonstrated chemical versatility allows a custom-made qRAS probe from a broad selection of fluorophores for different detection platforms (e.g., fluorescence microscopy, plate reader, flow cytometry).

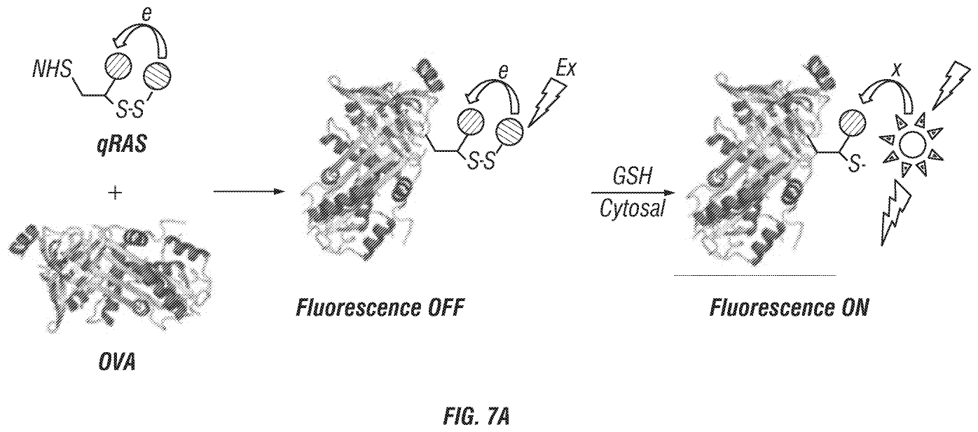

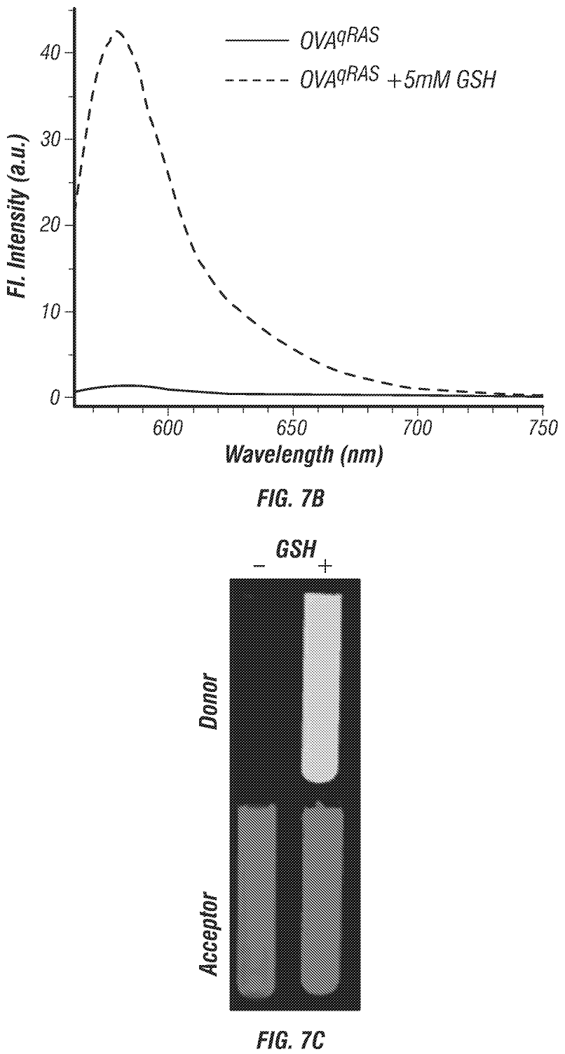

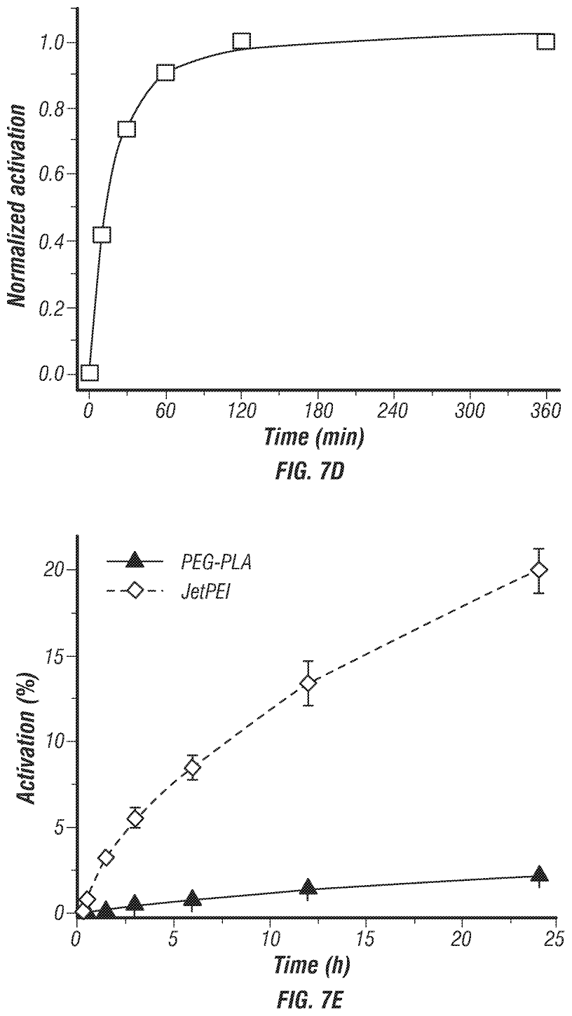

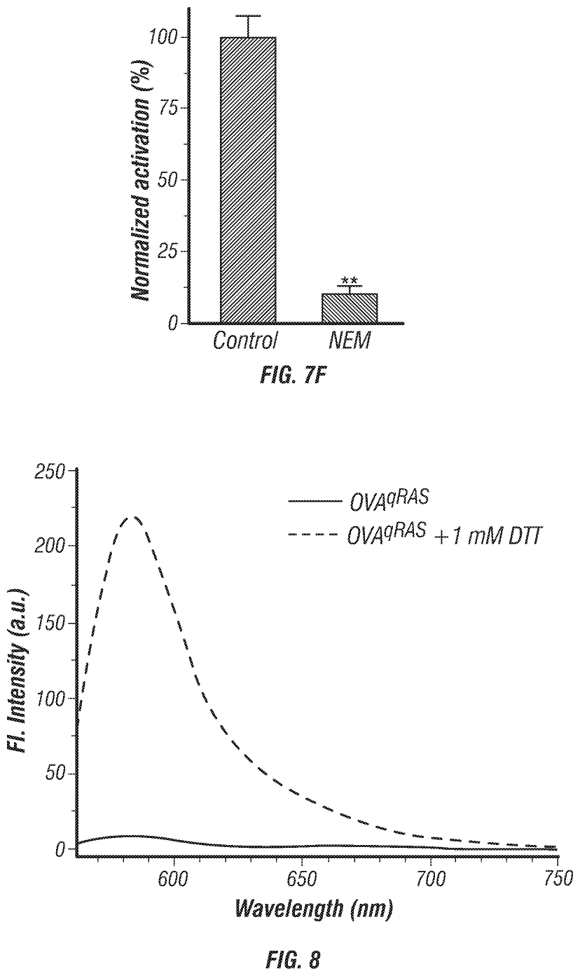

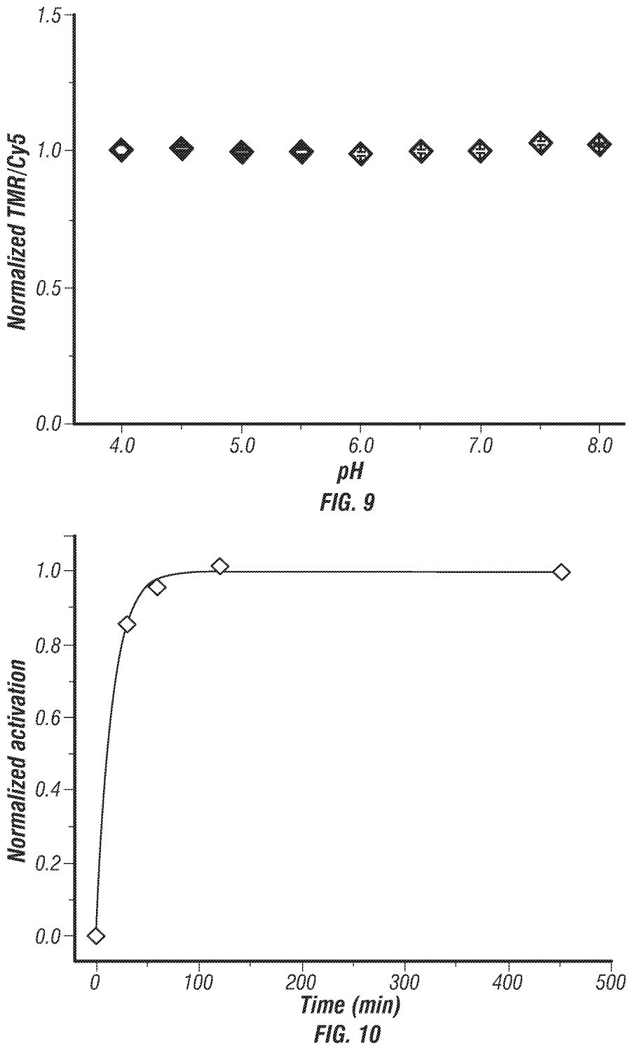

Next, it was examined whether qRAS-conjugated proteins are able to maintain the redox activation property. Ovalbumin was used as model protein and labeled by qRAS (OVA^{qRAS}, FIG. 7A). As shown in FIG. 7B, OVA^{qRAS} itself was almost nonfluorescent in the TMR channel as a result of efficient FRET quenching. After incubation with GSH (5 mM) for 1 hr, a substantial increase of emission intensity of TMR was observed (30-fold). Fluorescent images of OVA^{qRAS} solution with and without GSH addition demonstrated conspicuous on/off output in the donor channel (FIG. 7C). Similar fluorescence activation profile was detected when treating OVA^{qRAS} with dithiothreitol (DTT), a commonly used reducing agent for the disulfide bond (FIG. 8). The achieved fluorescence activation ratio (30-fold) offers a superior imaging window over reported FRET-based protein sensors (Xiong et al., 2014, Abraham et al., 2015, Xue et al., 2016 and Ogawa et al., 2009. Quantification of the kinetics of redox activation showed a half time (t_{1/2}) of 15 mins (FIG. 7D). In addition, the TMR/Cy5 ratio remained constant across a broad pH range from 4.0 to 8.0 (FIG. 9), which indicates that pH alone did not activate qRAS along the endocytic path.

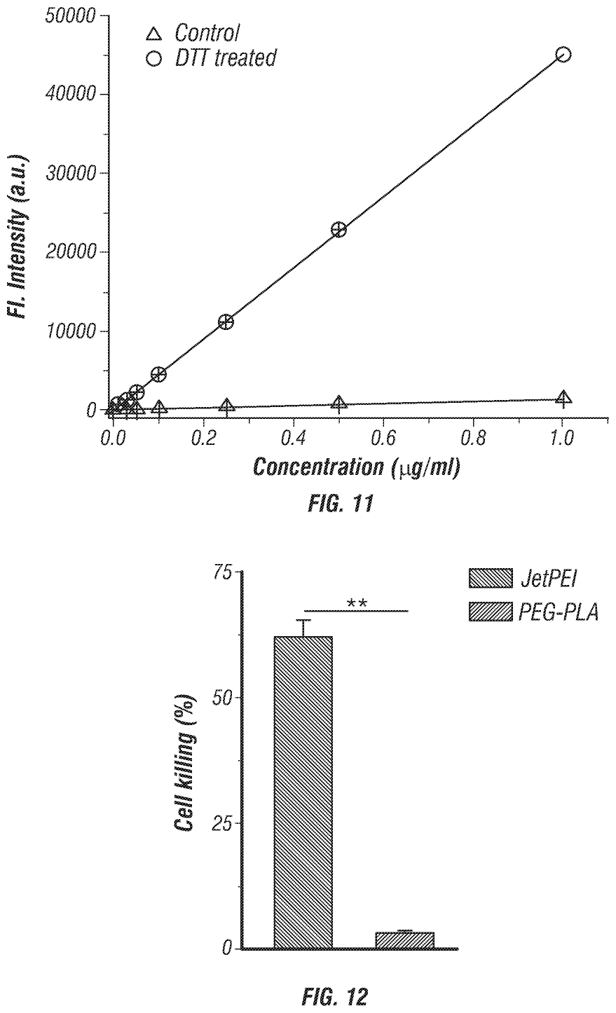

To examine the capability of OVA^{qRAS} for quantification of cytosolic delivery in living cells, the redox activation of OVA^{qRAS} was firstly demonstrated (FIG. 10) and a linear correlation between detected fluorescence signal and OVA^{qRAS} concentration on a plate reader (FIG. 11). Polyethylenimine (PEI) has been known to mediate endosomal escape of nucleic acids through the “proton sponge effect” (Benjaminsen et al., 2013 and Nel et al., 2009). A commercially available JetPEI was used in this study. PEG-b-poly(D, L-lactic acid) (PEG-PLA) was employed as a negative control due to its low transfection efficiency. Following co-incubation with OVA^{qRAS}, the fluorescence activation was monitored for 24 hr. Results showed that JetPEI led to significantly higher signal of activated OVA^{qRAS} over 24 hr when compared to PEG-PLA (FIG. 7E), consistent with the ability of JetPET for disrupting the endolysosomal membranes for cytosolic delivery. In addition, when replacing OVA^{qRAS} with a cytotoxic protein, ribonuclease A, JetPEI exhibited significantly stronger inhibitory effect on the proliferation of cancer cells over the PEG-PLA control (FIG. 12). To further demonstrate the GSH-specific activation of OVA^{qRAS}, we pretreated the A549 cells with N-ethylmaleimide (NEM), an irreversible and membrane-permeable sulfhydryl blocker. Data showed significant suppression of fluorescence signals (FIG. 7F), suggesting the redox activatable mechanism of OVA^{qRAS}. The ability to quantify cytosolic delivery efficiency on a plate reader offers a simple and high-throughput method for real-time monitoring of endolysosomal escape and cytosolic delivery of biomacromolecules.

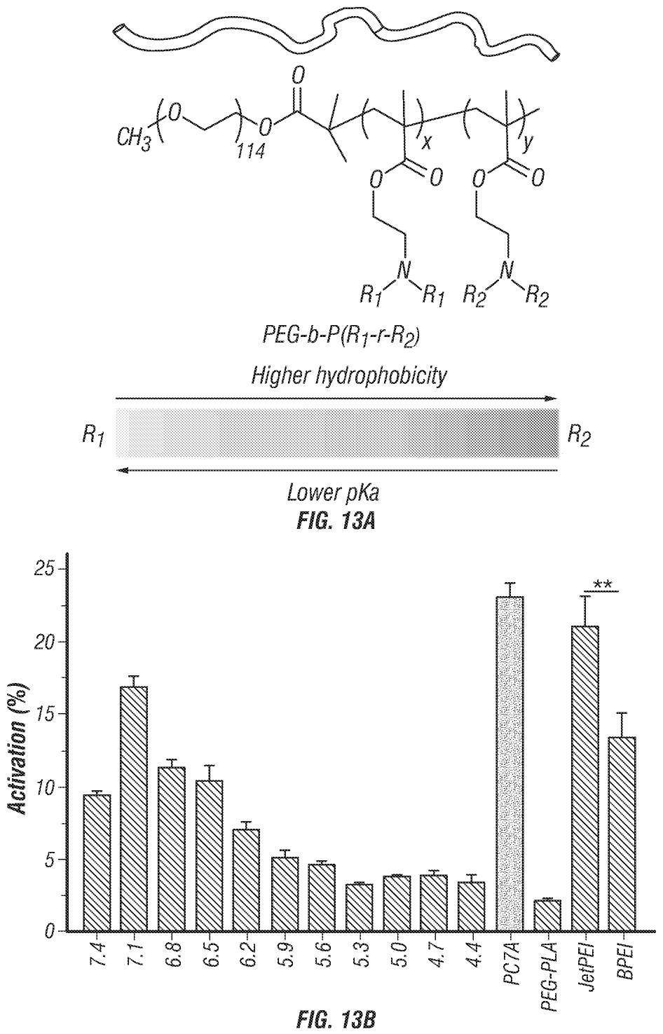

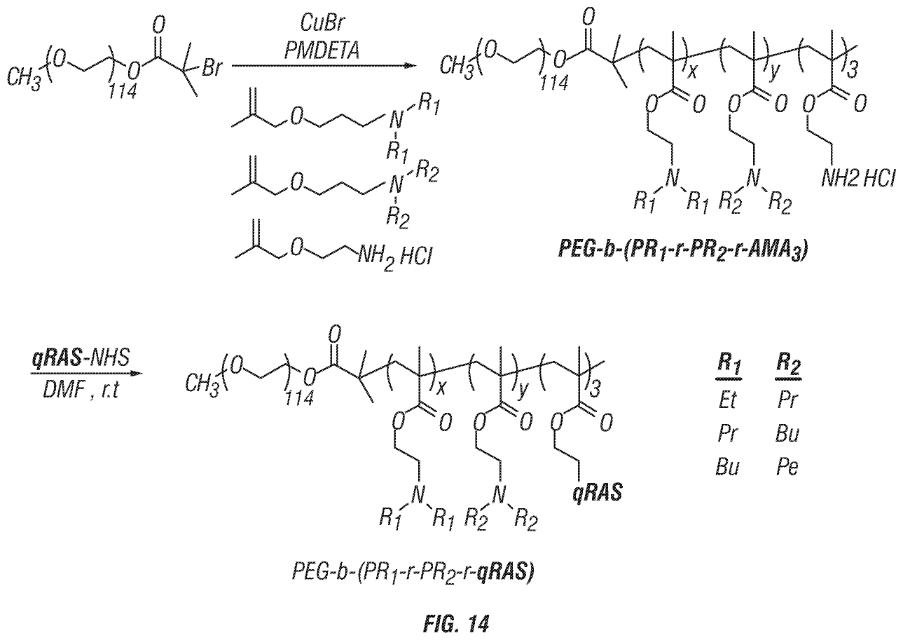

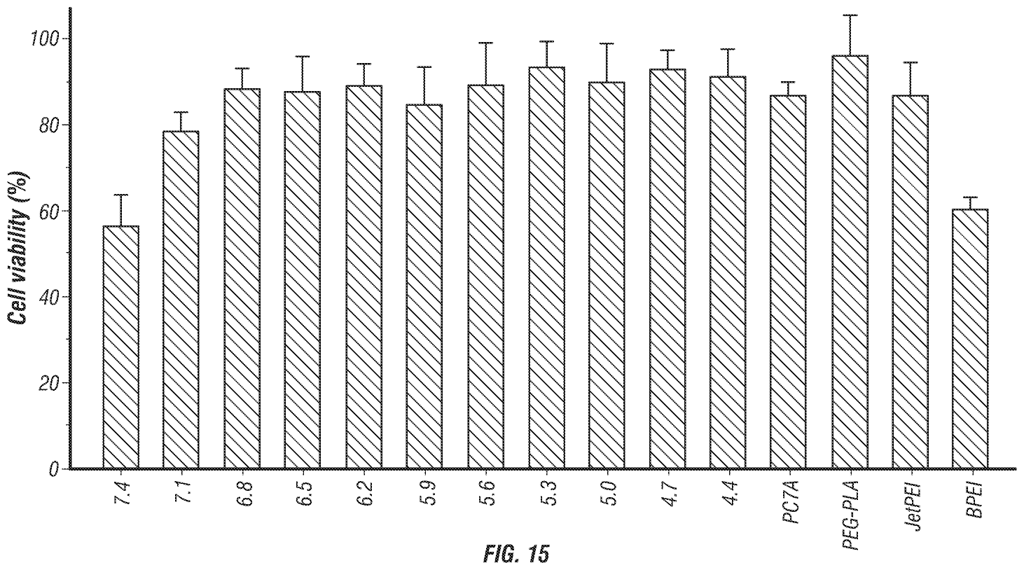

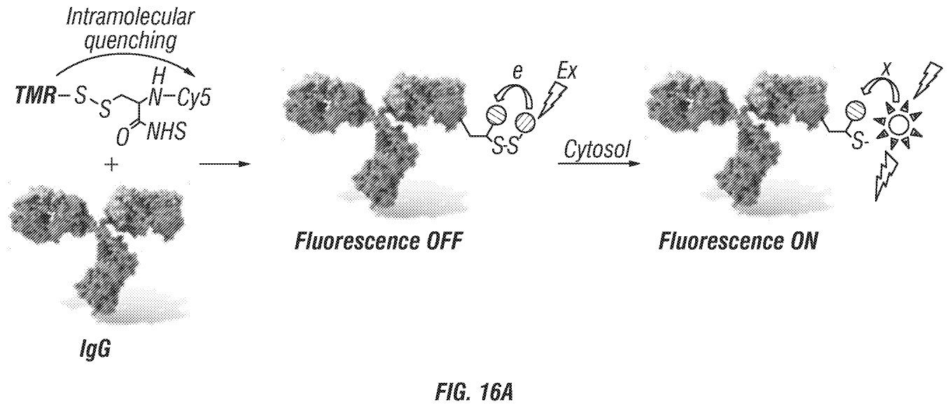

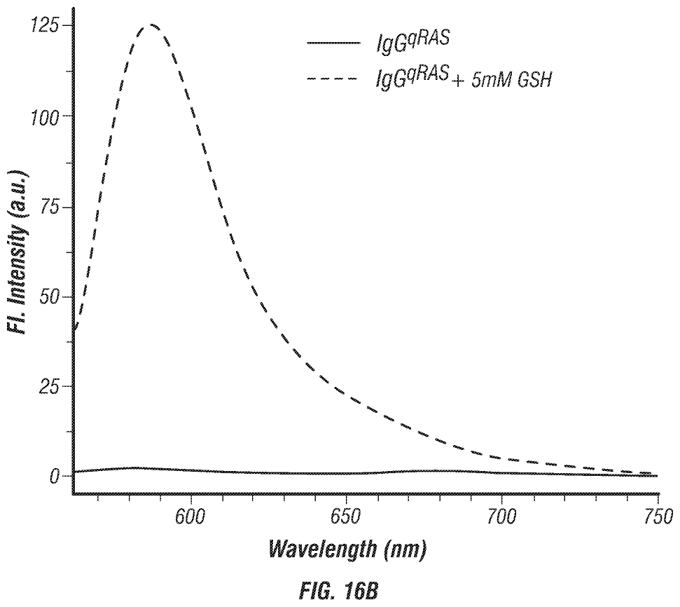

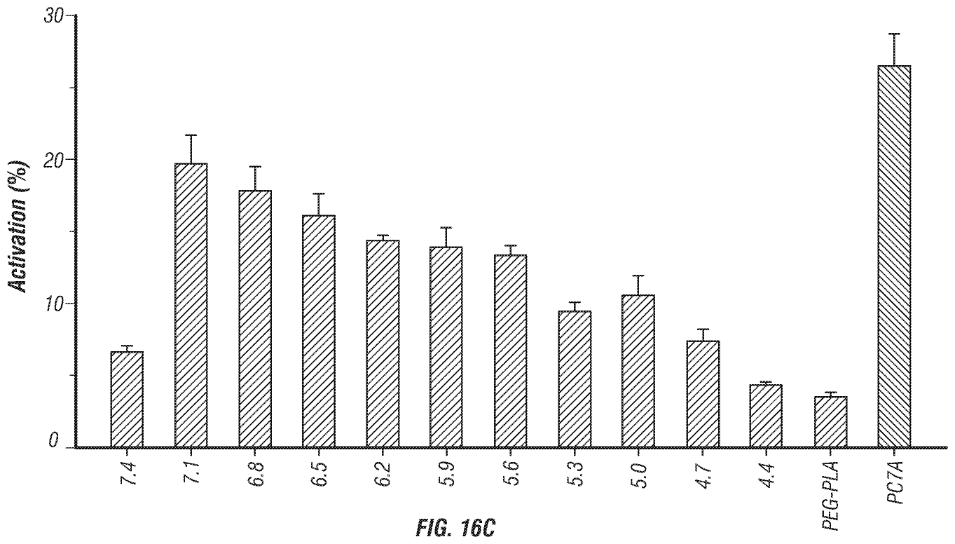

Previously, a library of ultra-pH-sensitive (UPS) copolymers with ionizable tertiary amine side chains has been synthesized. The library covers an entire physiologic range of endocytic pH (4.0-7.4) with 0.3 pH increments (FIGS. 13A & 14; Table 1) (Ma et al., 2014). In this study, the library of UPS copolymers was screened and evaluated their ability for cytosolic delivery of proteins. For the UPS copolymers with linear aliphatic side chains, higher pKₐ resulted in stronger activation signal of OVA^{qRAS} (FIG. 13B). The pH-specific strong buffer effect of UPS copolymers was previously reported at their pKₐ values (Wang et al., 2015). Results from this study indicate a pH selectivity where buffering the organelle pH at early stage of endocytic maturation is more effective for cytosolic delivery. In addition, it was observed that a UPS copolymer with a cyclic 7-membered ring on the side chain (PC7A, pKₐ=7.0) rendered the most efficient cytosolic delivery of proteins with minimal cytotoxicity (FIG. 15). The data also showed that JetPEI induced higher level of cytosol delivery of protein over branched PEI (BPEI, FIG. 13B), consistent with previous reports (Breunig et al., 2007, Wiseman et al., 2003 and Derouazi et al., 2004). Similar structural dependence of cytosolic delivery of UPS copolymers was also observed when immunoglobulin G was used as a model protein (FIG. 16), demonstrating the versatility of the qRAS probe for the labeling of different macromolecules to assess their cytosolic delivery in a high-throughput setting.

TABLE 1
Chemical compositions and physical
properties of UPS nanoparticles.

| Polymers | Composition^{a} | pKₐ^{b} | Dh (nm)^{c} | PDI^{c} |
|---|---|---|---|---|
| UPS_{7.4} | P(DEA₈₀) | 7.43 | 9.9 ± 1.1 | 0.25 ± 0.03 |
| UPS_{7.1} | P(DEA₅₈-DPA₄₂) | 7.05 | 28.4 ± 2.0 | 0.24 ± 0.01 |
| UPS_{6.8} | P(DEA₃₉-DPA₆₁) | 6.77 | 35.1 ± 4.8 | 0.24 ± 0.01 |
| UPS_{6.5} | P(DEA₂₁-DPA₇₉) | 6.45 | 45.1 ± 1.3 | 0.15 ± 0.01 |
| UPS_{6.2} | P(DPA₈₀) | 6.19 | 57.0 ± 2.7 | 0.18 ± 0.02 |
| UPS_{5.9} | P(DPA₆₀-DBA₂₀) | 5.89 | 68.1 ± 4.7 | 0.19 ± 0.03 |
| UPS_{5.6} | P(DPA₃₀-DBA₅₀) | 5.58 | 62.9 ± 6.5 | 0.15 ± 0.01 |
| UPS_{5.3} | P(DBA₈₀) | 5.31 | 71.8 ± 3.3 | 0.15 ± 0.06 |
| UPS_{5.0} | P(DBA₅₆-D5A₂₄) | 4.93 | 60.6 ± 5.4 | 0.19 ± 0.02 |
| UPS_{4.7} | P(DBA₂₈-D5A₅₂) | 4.65 | 65.2 ± 1.0 | 0.15 ± 0.01 |
| UPS_{4.4} | P(D5A₈₀) | 4.36 | 73.8 ± 3.1 | 0.18 ± 0.03 |
| PC7A | P(C7A₈₀) | 6.97 | 32.4 ± 2.0 | 0.30 ± 0.02 |

^{a}Only the composition of the PR segment is shown. The subscripts indicate the number of repeating unit for each monomer;
^{b}The apparent pKₐ values for UPS nanoparticles were determined by pH titration of polymer solutions using 4M NaOH in the presence of 150 mM NaCl. The maximum buffer pH corresponds to the apparent pKa of each copolymer;
^{c}The hydrodynamic diameter (Dh) and polydispersity index (PDI) were measured using dynamic light scattering analysis, mean ± S.D (n = 3).

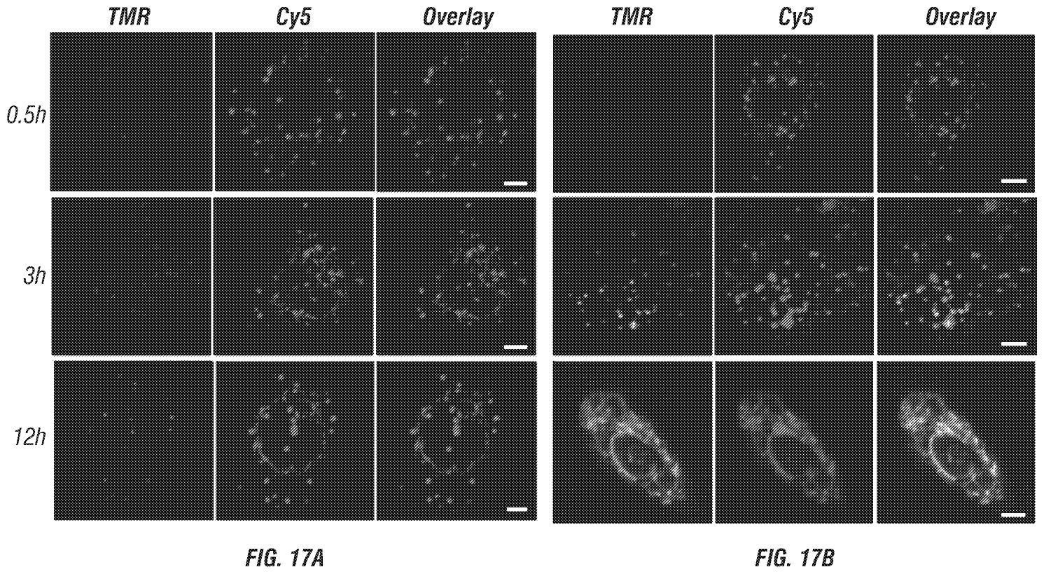

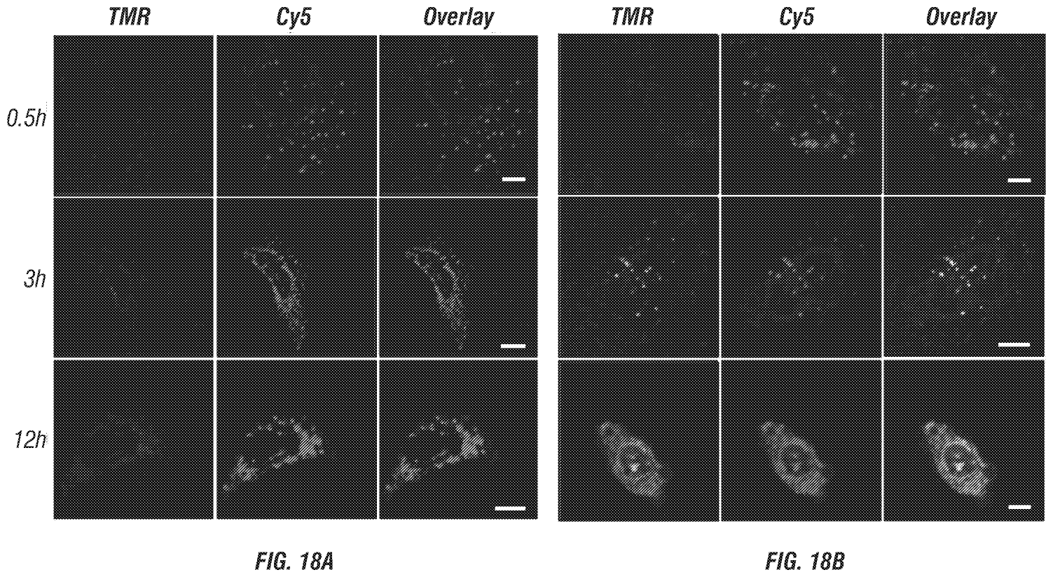

To evaluate whether qRAS is able to improve the accuracy of microscopic imaging of cytosolic delivery of macromolecules, confocal microscopy analysis of A549 cells incubated with IgG^{qRAS} was performed. PC7A and UPS_{4.4} copolymers with strong and weak cytosolic delivery efficiency, respectively, were employed for comparison. Cells treated with UPS_{4.4} showed a nominal donor TMR signal up to 12 hr indicative of minimal activation, while a punctate distribution of Cy5 signal confirmed endolysosomal entrapment of the protein (FIGS. 17A & 18A). In contrast, a dynamic pattern of endolysosomal disruption and cytosolic delivery of IgG was observed in PC7A treated cells. Emission intensity in the donor TMR channel was dramatically increased over time (FIGS. 17B & 18B). At 0.5 hr, no TMR signal was observed demonstrating the advantage of qRAS design in suppressing the signal from dose accumulation in the endocytic vesicles (IgG can still be tracked by the acceptor Cy5 signal). At 3 hr, a punctate distribution of TMR signal was observed, which implies that at early times, PC7A may disrupt endolysosomal membranes for GSH entry and activation of IgG^{qRAS} inside the vesicles. At 12 hr, endolysosomal escape of IgG proteins was pervasive as evidenced by broad TMR and Cy5 fluorescence spread throughout the cytoplasm (FIG. 18B). These data suggest that the qRAS design is able to overcome the challenge in cytosolic dilution and high signal intensity in endocytic vesicles for microscopic imaging of macromolecules using “always on” labels, leading to improved accuracy to study endolysosomal escape of macromolecules into the cell cytosol.

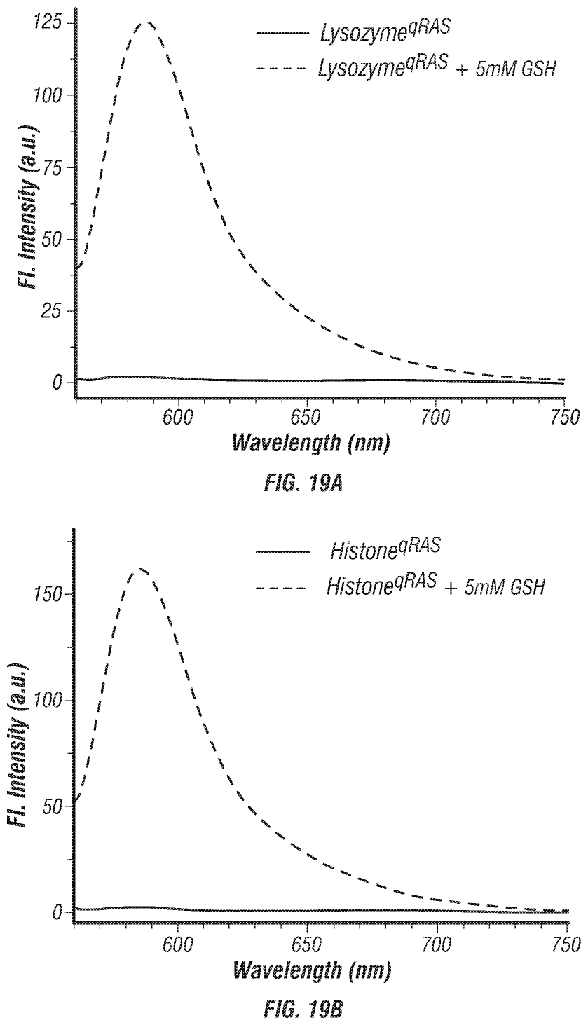

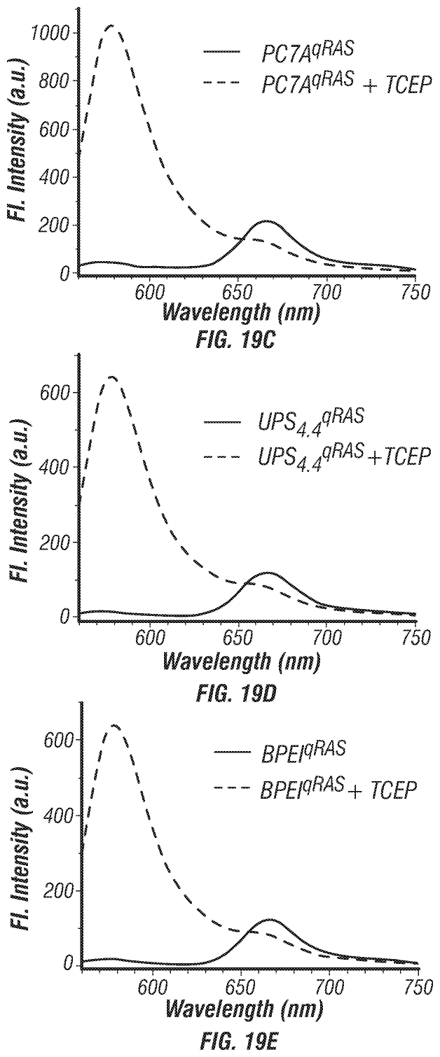

The qRAS probe described herein may be easily synthesized and conjugated to multiple classes of macromolecules (OVA, IgG, and additional proteins and polymers, FIG. 19). In these systems, macromolecules labeled with qRAS remain silent in the extracellular environment and intact endocytic vesicles, but can be dramatically activated in response to reducing environment of cytosol. The highly sensitive and specific cytosolic activation of the qRAS conjugates enables the quantitative assay of endolysosomal disruption on a microtiter plate allowing mechanistic investigation of key physicochemical parameters of existing nanocarriers as well as discover new compositions for cytosolic delivery of multiple classes of macromolecules.

### Example 2—Methods and Materials

### A. Materials

N-Hydroxysuccinimidal ester of tetramethyl rhodamine (NHS-TMR) was purchased from the Invitrogen Company. Cyanine 5-NHS ester was purchased from the Lumiprobe Corporation. Cysteamine 4-methoxytrityl resin was bought from EMD Millipore Corporation. N,N,N,N,N-Pentamethyldiethylenetriamine (PMDETA) was purchased from Sigma-Aldrich. PEG macroinitiator, MeO-PEG₁₁₄-Br, was prepared from 2-bromo-2-methyl propanoyl bromide and MeO-PEG₁₁₄-OH according to the procedure in literature (Zhou et al., 2011). Monomers such as 2-(diethylamino)ethyl methacrylate (DEA-MA) and 2-aminoethyl methacrylate (AMA) were purchased from Polyscience Company. Methacrylate monomers including 2-(dipropylamino) ethyl methacrylate (DPA-MA), 2-(dibutylamino) ethyl methacrylate (DBA-MA), 2-(dipentylamino) ethyl methacrylate (D5A-MA) and 2-(hexamethyleneimino) ethyl methacrylate (C7A-MA) were synthesized following previous publications (Zhou et al., 2012). AMA monomer was recrystallized twice with isopropanol and ethyl acetate (3:7) before use. JetPEI was bought from Polyplus-transfection. PEG₅₀₀₀-PLA₅₀₀₀ was purchased from Advanced Polymer Materials Inc. Amicon ultra-15 centrifugal filter tubes (Mw=10 K or 3,500 Da) were obtained from Millipore. Other solvents and reagents were used as received from Sigma-Aldrich or Fisher Scientific Inc.

### B. Synthesis of 2-(pyridin-2-yldisulfanyl)cysteine Hydrochloride

A solution of cysteine hydrochloride (3.16 g, 20.0 mmol) in methanol (40 mL) was added dropwise to a magnetically stirred mixture of 2,2′-dithiodipyridine (8.80 g, 40.0 mmol) and acetic acid (1.5 mL) in methanol (40 mL) (Chong and Hodges, 1981). After stirring at room temperature for 24 hr, the solvent was removed from the mixture by rotary evaporation. The product was dissolved in methanol and isolated by precipitating with diethyl ether. This washing/precipitation procedure was repeated 4 times to lead to a white product. Yield: 4.1 g (76.6%). ¹H NMR (400 MHz, DMSO-d₆, ppm): 8.72 (br s, 3H), 8.50 (ddd, J=4.8, 1.8, 0.9 Hz, 1H), 7.81 (td, J=7.7, 1.8 Hz, 1H), 7.71 (dt, J=8.2, 1.0 Hz, 1H), 7.29 (ddd, J=7.5, 4.9, 1.1 Hz, 1H), 4.17 (dd, J=7.1, 5.2 Hz, 1H), 3.43-3.27 (m, 2H). [M+H]⁺: 231.0 (calculated 231.3).

### C. Synthesis of TMR-SH

TMR-SH was synthesized following a previous report with minor modification (Gao et al., 2013). Firstly, cysteamine 4-methoxytrityl resin (18 mg) was pre-swelled in dimethylformamide (DMF) for 2 hr at room temperature. NHS-TMR (10.6 mg, 0.02 mmol) was dissolved in dry DMF (0.3 mL) and then added to the resin suspension with N,N-diisopropylethylamine (DIEA, 15 μL, 0.08 mmol). The reaction was proceeded in the dark for 24 hr. The resin was washed with DMF for 3 times followed by dichloromethane (DCM). After vacuum drying, the functionalized resin was treated with a mixture of trifluoroacetic acid (TFA, 0.2 mL) and DCM (0.4 mL). The solvent was evaporated at room temperature under vacuum. The resulting TMR-SH product was collected and its molecular weight was confirmed by HPLC-MS. [M+H]⁺: 490.0 (calculated 490.2).

### D. Synthesis of Small Molecule of qRAS

Compound 2-(pyridin-2-yldisulfanyl)cysteine hydrochloride (0.64 mg, 2.4 μM) and Cy5-NHS (1.54 mg, 2.5 μM) were dissolved in 0.2 mL of Ar saturated dry DMSO. Triethylamine (0.76 mg, 7.6 μM) was added. The resulting mixture was added into 100 mM phosphate-buffered saline (PBS, pH 7.6), then TMR-SH (1.23 mg, 2.5 μM) in Ar saturated DMSO was added in the mixture. The reaction was allowed at r.t. in the dark under Ar for 4 hr. The product was isolated using HPLC, and collected fractions were concentrated under reduced pressure to remove acetonitrile and lyophilized. [M+H]⁺: 1073.6 (calculated 1073.9).

This above compound was modified through the available carboxylic acids in order for further conjugation (Rybicka et al., 2010). In a typical procedure, the obtained compound (1.61 mg, 1.5 μM) was reacted with 1-ethyl-3-[3-dimethylaminopropyl]carbodiimide hydrochloride (0.96 mg, 5 μM) and N-hydroxysuccinimide (0.58 mg, 5 μM) in DMSO for 6 hr. The amine-reactive qRAS product was separated by chromatography using carboxymethyl-cellulose, the solvent was removed and stored at −80° C. [M+H]⁺: 1170.2 (calculated 1170.0). ¹H NMR (500 MHz, DMSO-d₆): δ=9.03 (d, J=6.0 Hz, 1H), 8.67 (s, 1H), 8.29-8.27 (m, 3H), 7.60 (d, J=7.4 Hz, 2H), 7.37 (dd, J=9.4, 7.2 Hz, 4H), 7.22 (q, J=8.1 Hz, 2H), 7.13-6.86 (m, 6H), 6.53 (t, J=12.3 Hz, 1H), 6.25 (d, J=13.8 Hz, 2H), 4.59-4.41 (m, 1H), 4.04 (t, J=7.4 Hz, 2H), 3.62 (d, J=6.4 Hz, 3H), 3.24-3.13 (m, 20H), 2.89 (s, 4H), 2.52 (s, 2H), 1.66-1.56 (m, 14H), 1.39 (q, J=7.8 Hz, 2H), 1.22 (t, 9H).

The position of Cy5 and TMR was switched and synthesized according to the same protocol. [M+H]⁺: 1170.1 (calculated 1170.0). ¹H NMR (500 MHz, DMSO): δ=9.25 (d, J=8.0 Hz, 1H), 8.73 (s, 1H), 8.37-8.27 (m, 3H), 7.60 (d, J=7.5 Hz, 2H), 7.37 (dd, J=9.5, 7.2 Hz, 4H), 7.22 (q, J=7.2 Hz, 2H), 7.12-6.92 (m, 6H), 6.51 (t, J=12.3 Hz, 1H), 6.24 (d, J=13.8 Hz, 2H), 4.79 (t, J=11.0 Hz, 1H), 4.05 (t, J=7.2 Hz, 2H), 3.56 (s, 3H), 3.28-3.11 (m, 20H), 2.77 (s, 4H), 2.05 (s, J=7.1 Hz, 2H), 1.66 (s, 14H), 1.57-1.43 (m, 2H), 1.32 (p, J=7.5, 7.0 Hz, 2H), 1.22 (t, 9H).

### E. Redox Activation of qRAS

To examine the fluorescence emission intensity of qRAS in response to a reducing stimulus, qRAS was firstly dissolved in methanol. Then 5 mM tris(2-carboxyethy)phosphine (TCEP) was added to trigger the cleavage of disulfide bond. The fluorescence spectra were recorded on a Hitachi Fluorometer with an excitation wavelength of 550 nm. In the meantime, the emission spectra of Cy5 was also measured as excited at 640 nm. The normalized ratio of the emission intensities of the TMR (F₅₇₅) and Cy5 (F₆₆₅) in response to redox cleavage was plotted over time.

### F. Conjugation and Characterization of qRAS to Ovalbumin (OVA)

To a solution of OVA (4.5 mg, 0.1 μM) in PBS (100 mM, pH 7.6) was added a solution of qRAS (0.32 mg, 0.3 μM) in 100 μL DMSO. The reaction mixture was vortexed, and then allowed to react at r.t. in the dark for 12 hr. The mixture was passed through a PD-10 gel filtration column to separate the product from unreacted qRAS and NHS byproduct. The obtained protein (OVA^{qRAS}) was lyophilized and stored at −80° C.

To assess its redox response, OVA^{qRAS} was added into a solution of PBS (pH 7.4) containing 5 mM GSH for 1 hr. The emission spectra of TMR was measured on a Hitachi Fluorometer with an excitation wavelength of 550 nm. In the meantime, the emission spectra of Cy5 was also recorded as excited at 640 nm. The fluorescent images of OVA^{qRAS} solution before and after the addition of GSH (5 mM) were obtained using the Maestro imaging system (CRI, Inc., Woburn, Mass.) with corresponding band pass excitation filter and long-pass emission filter according to the instrument manual.

### G. Syntheses of PEG-b-PR Block Copolymers

PEG-b-PR copolymers were synthesized by atom transfer radical polymerization (ATRP) following similar procedures previously reported (Tsarevsky and Matyjaszewski, 2007). PEG-b-PDBA (UPS_{5.3}) is used as an example to illustrate the procedure. First, DBA-MA (1.93 g, 8 mmol), PMDETA (23 μL, 0.1 mmol), and MeO-PEG₁₁₄-Br (0.5 g, 0.1 mmol) were charged into a polymerization tube. Then a mixture of 2-propanol (2 mL) and DMF (2 mL) was added to dissolve the monomer and initiator. After three cycles of freeze-pump-thaw to remove the oxygen, CuBr (15 mg, 0.1 mmol) was added into the polymerization tube under nitrogen atmosphere, and the tube was sealed in vacuo. The polymerization was carried out at 40° C. for 12 hr. After polymerization, the reaction mixture was diluted with 15 mL tetrahydrofuran (THF), and passed through a neutral Al₂O₃ column to remove the catalyst. The THF solvent was removed by rotovap. The residue was dialyzed against distilled water and lyophilized. After synthesis, the polymers were characterized by gel permeation chromatography (GPC).

### H. Preparation of Polymeric Nanoparticles

Nanoparticles were prepared following a solvent evaporation method. In the example of PEG-b-PDBA (UPS_{5.3}), 10 mg of the copolymer was dissolved in 1 mL THF and then added into 4 mL distilled water dropwise under sonication. The mixture was filtered 3 times to remove THF using the ultrafiltration system (Mw=10 KD). Then distilled water was added to adjust the polymer concentration to 10 mg/mL as a stock solution. The nanoparticles were characterized by dynamic light scattering for hydrodynamic diameter (Dh).

### I. Cells

A549 lung cancer cells were cultured in DMEM medium (Invitrogen, CA) supplemented with 5% fetal bovine serum (FBS), 100 IU/mL penicillin and 100 μg/mL streptomycin at 37° C. in 5% CO₂ atmosphere.

### J. High-Throughput Quantification Protocol

A549 cancer cells were seeded into 96-well black plate at a density of 10,000 cells per well and incubated for 24 hr. The polymer solution (100 μg/mL) and qRAS-labeled protein (OVA^{qRAS} or IgG^{qRAS}, 25 μg/mL) were co-incubated with cells for 40 min at 37° C. The cells were washed twice with PBS (pH 7.4) and cultured in complete culture medium. The fluorescence of TMR and Cy5 were measured on a Tecan fluorescent plate reader at different times. At 24 hr, 5 mM DTT was added to cleave the intracellular disulphide bond and completely release the TMR signal. The excitation and emission settings: TMR: Ex: 545 nm with bandwidth 9 nm, Em: 595 nm with bandwidth 20 nm; Cy5: Ex: 640 nm with bandwidth 9 nm, Em: 690 nm with bandwidth 20 nm.

The activation percentage (%) of OVA^{qRAS} was calculated using the equation described below:

Activation (%)=(F_{TMR}−F₀)/(F_{TMR,DTT}−F₀)*100

where F_{TMR} is the fluorescence intensity of TMR at different times, F_{TMR,DTT} is the fluorescence intensity of TMR after the addition of DTT at 24 hr, and F₀ is the background of TMR fluorescence.

For the N-ethylmaleimide (NEM) blocking experiments, the same procedures were used except that the cells were pretreated with 50 μM NEM for 30 min prior to the treatment with polymer and OVA^{qRAS}.

### K. Confocal Microscopy Analysis

A549 lung cancer cells were plated into glass bottom dishes (MatTek, MA) in 1 mL phenol red-free DMEM medium and were allowed to grow to 60-70% confluence. Cells were co-incubated with qRAS-labeled protein (OVA^{qRAS} or IgG^{qRAS}, 25 μg/mL) and UPS_{4.4} or PC7A for 40 min at a polymer concentration of 100 μg/mL. Then the medium was exchanged to complete DMEM medium and confocal images were acquired at different time points using the ZEISS LSM700 laser-scanning confocal microscope with a 60×objective lens. TMR and Cy5 were excited at 550 and 645 nm, respectively.

### L. Cytotoxicity Analysis of Polymers

A549 lung cancer cells were plated into 96-well plate at a density of 10,000 cells per well and incubated in the DMEM medium to allow cell growth for 24 hr. Then the cells were exposed to a series of polymers at 100 μg/mL for 40 min and washed twice with PBS (pH 7.4), and the fresh medium was added into plates. The cells were incubated for 48 hr before determination of cell viability. The cell viability was measured using an MTT assay (Tsarevsky and Matyjaszewski, 2007). Briefly, the cells were incubated with 0.5 mg/mL MTT solution for 4 hr, after which the medium was removed. Then 200 μL of DMSO was added into cell plates for OD determination at 570 nm using a microplate reader (SpectraMax M5, Molecular Devices, CA).

### M. In Vitro Efficacy of Cytotoxic Protein Delivery

Ribonuclease A was used as a cytotoxic protein to evaluate the cytosolic efficacy of JetPEI and PEG-PLA. Firstly, the lysine residues of proteins were reacted with cis-aconitic anhydride to convert the positively charged lysines into negatively charged carboxylate groups, increasing the binding ability with cationic polymers. Moreover, the modification is reversible in the acidic intracellular environment (e.g., endosomes and lysosomes), leading to the restoration of the biological activity of the modified proteins (Lee et al., 2009 and Wang et al., 2014). Typically, RNase A (10 mg) was dissolved in 0.1 M NaHCO₃ buffer solution (pH=9.0), cis-aconitic anhydride (50 mg) was added to the protein solution. After stirring for 2 hr, the mixture was purified using Amicon ultrafiltration tube (Mw=3,500 Da) for 3 times with distilled water. The RNase A-Aco was obtained after lyophilization.

To evaluate the efficacy, A549 cells were seeded into 96-well plates at a density of 10,000 cells per well. The cells were exposed to JetPEI (100 μg/mL) and PEG-PLA (100 μg/mL) with RNase A-Aco (5 μg/mL) for 40 min and washed twice with PBS (pH 7.4), and the fresh cell culture medium was added into plates. The cells were incubated for 48 hr before determination of cell viability using an MTT assay.

### N. Statistical Analysis

Statistical analysis was performed using Prism 5.0 (GraphPad). Data are expressed as means±S.D. Data were analyzed by Student's t test, and considered statistically significant if p<0.05. (*, p<0.05; **, p<0.01 unless otherwise indicated)

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Abraham et al., PLoS One, 10:e0134436, 2015.
Austin et al., Proc Natl Acad Sci USA, 102:17987-17992, 2005.
Austin-Ward and Villaseca, Rev. Med. Chil., 126(7):838-45, 1998.
Bareford and Swaan, Adv Drug Deliv Rev, 59:748-758, 2007
Belinsky and Jaiswal, Cancer Metast Rev. 12(2):103-117, 1993.
Benjaminsen et al., Mol Ther, 21:149-157, 2013;
Bey et al., Proc. Nat. Acad. Sci., 104(28):11832-11837, 2007.
Blanco et al., Cancer Res. 70(10):3896-3904, 2010.
Breunig et al., Proc Natl Acad Sci USA, 104:14454-14459, 2007.
Bukowski et al., Clin. Cancer Res., 4(10):2337-47, 1998.
Cao, et al., Anticancer Research 33(5):1785-1791, 2003.
Castanotto and Rossi, Nature, 457:426-433, 2009.
Chong and Hodges, J Biol Chem, 256:5071-5076, 1981.
Christodoulides et al., Microbiology, 144(Pt 11):3027-37, 1998.
Davidson et al., J Immunother., 21(5):389-98, 1998.
Deng et al., Biomaterials, 51:250-256, 2015.
Derouazi et al., Biotechnol Bioeng, 87, 537-545, 2004.
Dong et al., Cancer Res. 70(20):8088-8096, 2010.
Feener et al., J Biol Chem, 265:18780-18785, 1990.
Gao et al., Adv Mater, 25:3549-3553, 2013.
Gilleron et al., Nat Biotechnol, 31:638-646, 2013
Go and Jones, Biochim Biophys Acta, 1780:1273-1290, 2008.
Gu et al., Chem Soc Rev, 40:3638-3655, 2011
Hanibuchi et al., Int. J Cancer, 78(4):480-485, 1998.
Hellstrand et al., Acta Oncol., 37(4):347-353, 1998.
Holub et al., Biochemistry, 52:9036-9046, 2013.
Huang et al., Cancer Res. 72(12):3038-3047, 2012.
Hui and Hashimoto, Infect. Immun., 66(11):5329-36, 1998.
Jiang et al., Angew Chem Int Ed Engl, 54:506-510, 2015.
Johansson, Methods Mol Biol, 335:17-29, 2006.
Joseph et al., Oncol Res. 6(10-11):525-532, 1994.
Ju et al., Gene Ther., 7(19):1672-1679, 2000.
Kong and Mooney, Nat Rev Drug Discov, 6:455-463, 2007
Lachelt and Wagner, Chem Rev, 115:11043-11078, 2015
Lee et al., Angew Chem Int Ed Engl, 48:5309-5312, 2009.
Lee et al., J Am Chem Soc, 132:8828-8830, 2010.
Li et al., Clinical Cancer Research. 17(2):275-285, 2011.
Ma et al., J Am Chem Soc, 136:11085-11092, 2014.
Ma et al., Journal of Controlled Release. 200:201-211, 2015.
Malkinson et al., Cancer Res. 52(17):4752-4757, 1992.
Marin et al., BR. J CANCER. 76(7):923-929, 1997.
Mitchell et al., Ann. NYAcad. Sci., 690:153-166, 1993.
Mitchell et al., J Clin. Oncol., 8(5):856-869, 1990.
Morton et al., Arch. Surg., 127:392-399, 1992.
Nel et al., Nat Mater, 8:543-557, 2009.
Ogawa et al., ACS Chem Biol, 4:535-546, 2009.
Onyango et al., Angew Chem Int Ed Engl, 54:3658-3663, 2015;
Pack et al., Nat Rev Drug Discov, 4:581-593, 2009
Pannecouque et al., Nat Protoc, 3:427-434, 2008.
PCT Application Publication No. WO 1996/033988
PCT Application Publication No. WO 2004/045557
PCT Application Publication No. WO 2006/128120
PCT Application Publication No. WO 2008/066294
PCT Application Publication No. WO 2009/105166
Pietras et al., Oncogene, 17(17):2235-49, 1998.
Qin et al., Proc. Natl. Acad. Sci. USA, 95(24):14411-14416, 1998.
Ravindranath and Morton, Intern. Rev. Immunol., 7: 303-329, 1991.
Rietsch and Beckwith, Annu Rev Genet, 32:163-184, 1998.
Rosenberg et al., Ann. Surg. 210(4):474-548, 1989.
Rosenberg et al., N. Engl. J. Med., 319:1676, 1988.
Roy et al., Nat Methods, 5:507-516, 2008.
Rybicka et al., Proc Natl Acad Sci USA, 107:10496-10501, 2010.
Sahay et al., Nat Biotechnol, 31:653-658, 2013.
Santra et al., J Am Chem Soc, 133:16680-16688, 2011.
Schafer and Buettner, Free Radic Biol Med, 30:1191-1212, 2001.
Tsarevsky and Matyjaszewski, Chem Rev, 107:2270-2299, 2007.
Wang et al., Angew Chem Int Ed Engl, 53:2893-2898, 2014.
Wang et al., Nat Commun, 6:8524, 2015.
Whitehead et al., Nat Rev Drug Discov, 8:129-138, 2009.
Wiseman et al., Gene Ther, 10:1654-1662, 2003;
Xiong et al., Nat Commun, 5:3992, 2014;
Xue et al., J Am Chem Soc, 138:5258-5261, 2016.
Zhou et al., Angew Chem Int Ed Engl, 50:6109-6114, 2011.
Zhou et al., J Am Chem Soc, 134:7803-7811, 2012.
U.S. Pat. No. 5,739,169
U.S. Pat. No. 5,801,005
U.S. Pat. No. 5,824,311
U.S. Pat. No. 5,830,880
U.S. Pat. No. 5,846,945
U.S. Pat. No. 6,262,095

1. A compound of the formula: 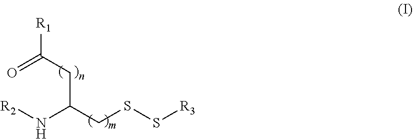
wherein:
R₁ is a first label, amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, an amine reactive group; a thiol reactive group, an organic polymer, a nucleic acid sequence, a peptide, or a protein;
R₂ is a first label, hydrogen, alkyl_{(C≤8)}, substituted alkyl_{(C≤8)}, an organic polymer, a nucleic acid sequence, a peptide, or a protein;
R₃ is a second label; and
m and n are each independently 0, 1, 2, or 3;
provided that either R₁ or R₂ is a first label.

2. The compound of claim 1 further defined as: 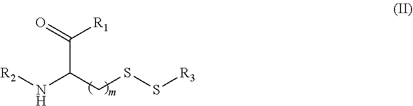
wherein m, R₁, R₂, and R₃ are as defined above.

3. The compound of claim 2 further defined as: 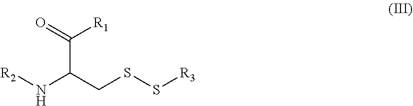
wherein R₁, R₂, and R₃ are as defined above.

4. The compound according to claim 1, wherein R₂ or R₃ is a fluorescent dye.

5. The compound of claim 4, wherein R₂ or R₃ are fluorescent dyes.

6-9. (canceled)

10. The compound of claim 4, wherein R₂ or R₃ is a xanthene dye.

11. (canceled)

12. The compound of claim 4, wherein R₂ or R₃ is a cyanine dye, a coumarin dye or a BODIPY dye.

13-14. (canceled)

15. The compound according to claim 1, wherein R₂ or R₃ is a fluorescent quencher.

16-17. (canceled)

18. The compound according to claim 1, wherein m is 1 or 2.

19. The compound according to claim 1, wherein n is 0 or 1.

20. The compound according to claim 1, wherein R₁ is hydroxy and thereby forms a carboxylic acid.

21. The compound according to claim 1, wherein R₁ is an amine reactive group.

22. (canceled)

23. The compound according to claim 1, wherein R₁ is an organic polymer, a nucleic acid sequence, a peptide, or a protein.

24-27. (canceled)

28. The compound according to claim 1, wherein the compound is further defined as: 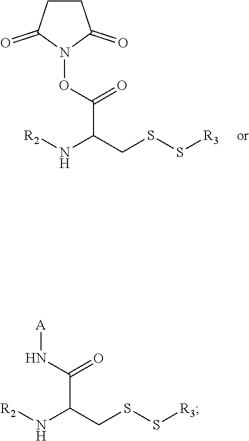
wherein:
R₂ and R₃ are each independently a fluorescent dye or a fluorescent quencher; and
A is a peptide, protein, or nucleic acid.

29-31. (canceled)

32. A composition comprising:
(a) a compound according to claim 1; and
(b) a polymer;
wherein the polymer forms a micelle.

33. The composition of claim 32, wherein the polymer is a compound of the formula: 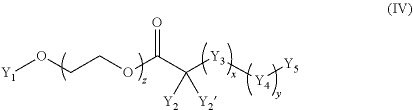
wherein:
Y₁ is hydrogen, alkyl_{(C≤12)}, cycloalkyl_{(C≤12)}, substituted alkyl_{(C≤12)}, substituted cycloalkyl_{(C≤12)}, a cell targeting moiety, or a conjugating group;
n is an integer from 1 to 500;
Y₂ and Y₂′ are each independently selected from hydrogen, alkyl_{(C≤12)}, cycloalkyl_{(C≤12)}, substituted alkyl_{(C≤12)}, or substituted cycloalkyl_{(C≤12)};
Y₃ is a group of the formula: 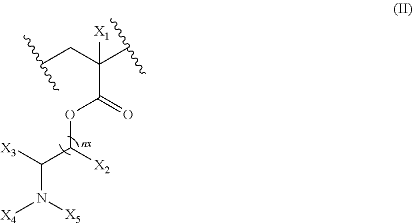
wherein:
nₓ is 0-3;
X₁, X₂, and X₃ are each independently selected from hydrogen, alkyl_{(C≤12)}, cycloalkyl_{(C≤12)}, substituted alkyl_{(C≤12)}, or substituted cycloalkyl_{(C≤12)}; and
X₄ and X₅ are each independently selected from alkyl_{(C≤12)}, cycloalkyl_{(C≤12)}, or a substituted version of any of these groups, or X₄ and X₅ are taken together and are alkanediyl_{(C≤12)}, alkoxydiyl_{(C≤12)}, alkylaminodiyl_{(C≤12)}, or a substituted version of any of these groups;
x is an integer from 1 to 150;
Y₄ is a group of the formula: 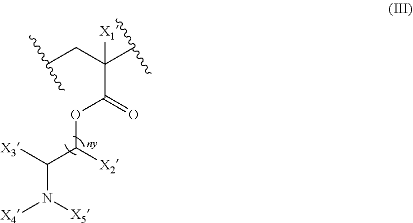
wherein:
n_{y} is 0-3;
X₁′, X₂′, and X₃′ are each independently selected from hydrogen, alkyl_{(C≤12)}, cycloalkyl_{(C≤12)}, substituted alkyl_{(C≤12)}, or substituted cycloalkyl_{(C≤12)}; and
X₄′ and XV′ are each independently selected from hydrogen, alkyl_{(C≤12)}, cycloalkyl_{(C≤12)}, acyl_{(C≤12)}, substituted alkyl_{(C≤12)}, substituted cycloalkyl_{(C≤12)}, substituted acyl_{(C≤12)}, a dye, or a quencher;
y is an integer from 0 to 20; and
Y₅ is hydrogen, halo, hydroxy, alkyl_{(C≤12)}, or substituted alkyl_{(C≤12)}.

34. A method of determining the redox state of a composition comprising:
(a) contacting the composition with a compound or pharmaceutical composition according to claim 1; and
(b) measuring for a signal from the each of the labels of the compound which indicates a reducing environment.

35. The method of claim 34, wherein the composition is a cell.

36. The method of claim 34, wherein the composition is a human body.

37. The method of claim 34, wherein the composition is a composition outside of a living organism.

38. The method of claim 34, wherein one of the label allows the imaging of the location of the compound in the composition.
